(19) United States
(12) Patent Application Publication
(10) Pub. No.: US 2008/0125609 A1
(43) Pub. Date: May 29, 2008

(54) Compounds for Organic Electronic Devices

(75) Inventors: Horst Vestweber, Gilersberg-Winterscheid (DE); Susanne Heun, Bad Soden (DE); Amir Parham, Frankfurt (DE); Philipp Stossel, Frankfurt am Main (DE); Holger Heil, Darmstadt (DE); Rocco Fortte, Frankfurt (DE)

Correspondence Address: CONNOLLY BOVE LODGE & HUTZ, LLP, P O BOX 2207, WILMINGTON, DE, 19899, US

(73) Assignee: Merck Patent GmbH, Darmstadt (DE)

(21) Appl. No.: 11/720,574

(22) PCT Filed: Dec. 1, 2005
(86) PCT No.: PCT/EP05/12807

§ 371 Date: May 31, 2007

(30) Foreign Application Priority Data
Dec. 1, 2004 (EP) 04028407.7

(51) Int. Cl. H01J 1/62; C07C 211/54

(52) U.S. Cl. 564/427; 564/428; 428/690

(57) ABSTRACT

The invention relates to a compound of the formula (1),

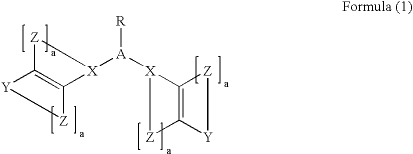

and to the use of the compound in organic electroluminescent devices.

The present invention relates to new types of compounds and to their use in organic electroluminescent devices.

The use of semiconductive organic compounds in organic electroluminescent devices (OLEDs) is just starting to be introduced onto the market. The general structure of such devices is described, for example, in U.S. Pat. No. 4,539,507, U.S. Pat. No. 5,151,629, EP 0676461 and WO 98/27136. Devices comprising simple OLEDs have already been introduced onto the market as demonstrated by the car radios from Pioneer, is the mobile telephones from Pioneer and SNMD or a digital camera from Kodak with an organic display. Further products of this type will shortly be introduced. However, these devices still exhibit considerable problems which are in need of urgent improvement:
1. The operative lifetime, especially in the case of blue emission, is still too low, so that it has been possible to date to commercially realize only simple applications.
2. The efficiency has been improved in the last few years, but is still too low specifically for fluorescent OLEDs and has to be improved. One cause of this might be a low photoluminescence quantum yield for many of the materials typically used as blue emitters.
3. Many blue-emitting compounds which contain both aromatic amines and double bond systems are thermally unstable and decompose in the course of sublimation or in the course of vapour depositions. As a result, it is possible to use these systems only with great losses, if at all, and with a high level of technical complexity, and continuous long-term production is not possible.
4. Many blue-emitting compounds which contain double bond systems exhibit isomerization from the trans to the cis configuration, especially upon heating or upon electronic excitation. It is also known that cis-stilbene derivatives react photochemically to give dihydrophenanthrenes which can then react further oxidatively to give phenanthrenes. This can result in poorer reproducibility of the device, since these reaction products have different electronic and photochemical properties from the reactants.

The closest prior art may be specified as the use of particular arylvinylamines which do not have further substitution on the double bond of the stilbene-like system by Idemitsu (for example WO 04/013073, WO 04/016575, WO 04/018587). Using these, good lifetimes are reported for deep blue emissions. However, these results are greatly dependent upon the host material used, so that the cited lifetimes cannot be compared as absolute values, but rather always only in the case of use in an optimized host system. Moreover, these compounds are thermally unstable and cannot be evaporated without decomposition, which therefore entails a high level of technical complexity for the vapour deposition and thus a distinct technical disadvantage. A further disadvantage is the emission colour of these compounds. While Idemitsu reports deep blue emission (CIE y coordinates in the range of 0.15-0.18), it has not been possible to reproduce these colour coordinates in simple devices according to the prior art. On the contrary, green-blue emission with CIE y coordinates in the range of 0.30-0.35 is obtained here. It is not apparent how blue emission can be obtained with these compounds.

We suspect that the double bonds of the compounds described in the literature might be responsible at least for some of the abovementioned problems. For instance, the double bond might tend to polymerize in the course of heating (for example in the course of sublimation to purify the compounds or in the course of vapour deposition in the production of the device), or might isomerize from the trans to the cis configuration in the excited state in the course of operation of the device. Even in the event of identical substitution, in which case the consequence of isomerization is not so severe because the product has the same structure as the reactant, it deactivates the excitation energy of the molecule in a non-radiative manner. These side reactions might therefore reduce the efficiency or the lifetime of the organic electronic device. Moreover, these side reactions are possibly responsible for the low thermal stability of these compounds.

There is thus still a need for blue-emitting compounds which lead to good efficiencies and simultaneously to high lifetimes in organic electroluminescent devices, and which are thermally stable and can thus be processed in a technically unproblematic manner. It has now been found that, surprisingly, organic electroluminescent devices which comprise certain compounds, detailed below, whose double bonds cannot exhibit cis-trans-isomerization by virtue of the chemical structure as blue-emitting dopants in a host material have distinct improvements over the prior art. It is possible with these materials to simultaneously obtain high efficiencies and long lifetimes. In other functions, too, these materials in organic electroluminescent devices and further organic electronic devices exhibit good properties. Moreover, these compounds, unlike materials according to the prior art, can be sublimed and applied by vapour deposition without noticeable decomposition and are therefore distinctly easier to handle than materials according to the prior art. These compounds and their use in organic electronic devices therefore form part of the subject-matter of the present invention.

The invention provides compounds of the formula (1),

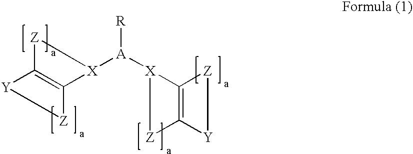

where the symbols used are:
A is the same or different at each instance and is N, P or P═O;
X, Y are the same or different at each instance and are each an aromatic or heteroaromatic system which has 5 to 60 aromatic ring atoms that may be substituted by one or more R¹ radicals;
R is the same or different at each instance and is a straight-chain alkyl group having 1 to 40 carbon atoms or a branched or cyclic alkyl group having 3 to 40 carbon atoms, each of which may be substituted by one or more R¹ radicals, in which one or more nonadjacent CH₂ groups may be replaced by —R²C═CR²—, —C≡C—, P(═O)(R²), SO, SO₂, Si(R²)₂, Ge(R²)₂, Sn(R²)₂, C═O, O═S, C═Se, C═NR², —O—, —S— or —CONR²—, and in which one or more hydrogen atoms may be replaced by F, Cl, Br, I, CN or N₂, or an aromatic or heteroaromatic system which has 5 to 40 aromatic ring atoms and may be substituted by one or more R² radicals, or an aryloxy or heteroaryloxy group which has 5 to 40 aromatic ring atoms and may be substituted by one or more R² radicals, or a combination of two, three or four of these systems; or R is a group of the formula (2),

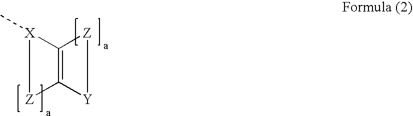

where the symbols are each defined as described above and below, and the dashed bond symbolizes the attachment to A;
Z is the same or different at each instance and is a bivalent group C(R¹)₂, C═O, C[═C(R¹)₂], Si(R¹)₂, O, S═O, SO₂, NR, BR¹, PR¹, PR¹O, C(R¹)₂—C(R¹)₂, C(R¹)₂—NR¹, C(R¹)₂—C(R¹)₂—C(R¹)₂ or C(R¹)₂—O—C(R¹)₂, where Z, apart from to the double bond, also bonds to the Y group or to the X group on the same double bond, preferably in the ortho position or peri position, and thus forms a further cyclic ring system;
R¹ is the same or different at each instance and is H, F, Cl, Br, I, CN, NO₂, a straight-chain alkyl-, alkoxy- or thioalkoxy group having 1 to 40 carbon atoms or a branched or cyclic alkyl, alkoxy or thioalkoxy group which has 3 to 40 carbon atoms, and may be substituted in each case by one or more R² radicals in which one or more nonadjacent CH₂ groups may be replaced by —R²C═CR²—, —C≡C—, P(═O)(R²), SO, SO₂, Si(R²)₂, Ge(R²)₂, Sn(R²)₂, C═O, C═S, C═Se, C═NR², —O— —S— or —CONR²— and in which one or more hydrogen atoms may be replaced by F, Cl, Br, I, CN or NO₂, or an aromatic or heteroaromatic system which has 5 to 40 aromatic ring atoms and may be substituted by one or more R² radicals, or an aryloxy- or heteroaryloxy group which has 5 to 40 aromatic ring atoms and may be substituted by one or more R² radicals, or a combination of two, three or four of these systems; in this radical, two or more substituents R¹ together may also form a mono- or polycyclic aliphatic ring system;
R² is the same or different at each instance and is H or an aliphatic or aromatic hydrocarbon radical having 1 to 20 carbon atoms;
a is the same or different at each instance and is 0 or 1, with the proviso that at least one index is a=1 per double bond, where a=0 means that, instead of Z, an R¹ group is bonded to the double bond and to X or Y.

Even if it is evident from the description, it should be pointed out explicitly once again here that the R¹ radicals can also form a ring system with one another and can thus in particular form a spiro system.

An aryl group in the context of this invention contains 6 to 40 carbon atoms; a heteroaryl group in the context of this invention contains 2 to 40 carbon atoms and at least 1 heteroatom, with the proviso that the sum of carbon atoms and heteroatoms adds up to at least 5. The heteroatoms are preferably selected from N, O and/or S. An aryl group or heteroaryl group refers either to a simple aromatic cycle, i.e. benzene, or a simple heteroaromatic cycle, for example, pyridine, pyrimidine, thiophene, etc., or a fused aryl or heteroaryl group.

An aromatic ring system in the context of this invention contains 6 to 40 carbon atoms in the ring system. A heteroaromatic ring system in the context of this invention contains 2 to 40 carbon atoms and at least one heteroatom in the ring system, with the proviso that the sum of carbon atoms and heteroatoms adds up to at least 5. The heteroatoms are preferably selected from N, O and/or S. In the context of this invention, an aromatic or heteroaromatic ring system shall be understood to mean a system which does not necessarily contain only aryl or heteroaryl groups, but in which a plurality of aryl or heteroaryl groups may also be interrupted by a short non-aromatic unit (fewer than 10% of the atoms other than H, preferably fewer than 5% of the atoms other than H), for example an sp³-hybridized carbon, nitrogen or oxygen atom. For example, aromatic ring systems in the context of this invention should also be understood to mean systems such as 9,9′-spirobifluorene, 9,9-diarylfluorene, triarylamine, diaryl ether, etc. In this case, a portion of the aromatic or heteroaromatic ring system may also be a fused group.

In the context of the present invention, a C₁- to C₄₀-alkyl group in which individual hydrogen atoms or CH₂ groups may also be substituted by the above-mentioned groups is more preferably understood to mean the methyl, ethyl, n-propyl, i-propyl, n-butyl, i-butyl, s-butyl, t-butyl, 2-bethylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals. A C₁- to C₄₀-alkoxy group is more preferably understood to mean methoxy, ethoxy, n-propoxy, i-propoxy, n-butoxy, i-butoxy, s-butoxy, t-butoxy or 2-methylbutoxy. An aromatic or heteroaromatic ring system which has 5-40 aromatic ring atoms, and may also be substituted in each case by the abovementioned R radicals and which may be attached via any positions to the aromatic or heteroaromatic is understood in particular to mean groups which are derived from benzene, naphthalene, anthracene, phenanthrene, pyrene, chrysene, perylene, fluoranthene, naphthacene, pentacene, benzpyrene, biphenyl, biphenylene, terphenyl, terphenylene, fluorene, spirobifluorene, dihydrophenanthrene, dihydropyrene, tetrahydropyrene, cis- or trans-indenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazineimidazole, quinoxalineimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzpyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

In a preferred embodiment of the invention R is a group of the formula (2).

Preference is given to the compounds of the formula (1) for which:
A is N or P at each instance;
X is the same or different at each instance and is a bivalent aryl or heteroaryl group which has 5 to 25 aromatic ring atoms, and may be substituted by one, two, three or four R¹ radicals;
Y is the same or different at each instance and is a monovalent aryl or heteroaryl group which has 5 to 25 aromatic ring atoms, and may be substituted by one, two, three or four R¹ radicals;
R is a group of the abovementioned formula (2) at each instance;
Z is the same or different at each instance and is C(R¹)₂, SO₂, BR¹, P(R¹)O, C(R¹)₂—C(R¹)₂, C(R¹)₂—NR¹;
a is equal to 0 or 1 at each instance, where one index is a=0 and the other index is a=1 on each double bond;
R¹ and R² are each as defined above.

Particular preference is given to compounds of the formula (1) for which:
A is N at each instance;
X is the same or different at each instance and is a bivalent aryl group which has 6 to 16 carbon atoms, and may be substituted by one or two R¹ radicals;
Y is the same or different at each instance and is a monovalent aryl group which has 6 to 16 carbon atoms, and may be substituted by one, two or three R¹ radicals;
Z is the same or different at each instance and is C(R¹)₂, P(R¹)O or C(R¹)₂—C(R¹)₂;
R, R¹, R² and a are each as defined above.

This preference applies in particular when the compound of the formula (1) is used as a blue-emitting dopant. In other functions, in the organic electronic device another selection of the groups may be preferred, especially of the A and Z groups.

Preference is further given to compounds of the formula (1) in which all symbols X represent the same aromatic or heteroaromatic system, more preferably benzene or naphthalene, most preferably benzene. Particular preference is also given to all symbols X having identical substitution.

Preference is likewise given to compounds of the formula (1) in which all symbols Y represent the same aromatic or heteroaromatic system, more preferably benzene or naphthalene, most preferably benzene. Particular preference is also given to all symbols Y having identical substitutions.

Preference is further given to compounds of the formula (1) in which all units Z are each selected identically and also bond to the same positions on X and Y in each case.

Particular preference is given to compounds of the formula (1), which have a symmetrical structure and which have a threefold rotational axis, especially those in which all units X and all units Y and Z and all substituents R¹ and R² are each selected identically.

These preferences arise in particular from the easier synthetic obtainability of the symmetrically structured compounds. However, more complicated synthetic methods in principle also make obtainable the unsymmetrically substituted compounds.

Preference is further given to the compounds of the formula (1) which have no benzylic protons. This applies in particular to radicals on the Z group. When the Z group is C(R¹)₂ or O(R¹)₂—C(R¹)₂, R¹ is thus preferably not H. The reason for this preference is the comparatively high reactivity of benzylic protons so that the presence of benzylic protons might possibly lead to undesired side reactions in device production and operation.

A particularly preferred embodiment of the invention is that of compounds of the formula (3)

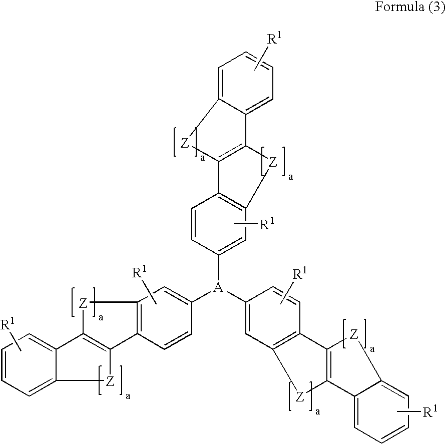

where A, Z, R¹ and a are each defined as described above, where the maximum number of substituents R¹ corresponds to the number of substitutable hydrogen atoms.

For particularly preferred compounds of the formula (3), the same preferences for the symbols A and Z apply as detailed above for compounds of the formula (1).

Examples of preferred compounds of formula (1) or formula (3) are the structures (1) to (30) depicted below, which may be substituted by R¹ or unsubstituted.

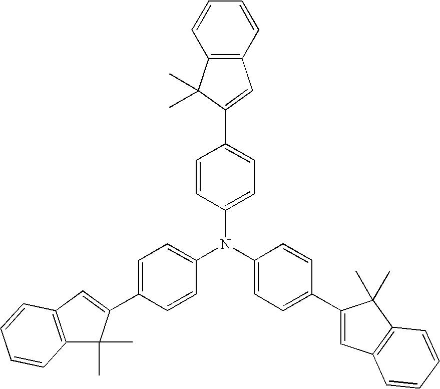

(1)

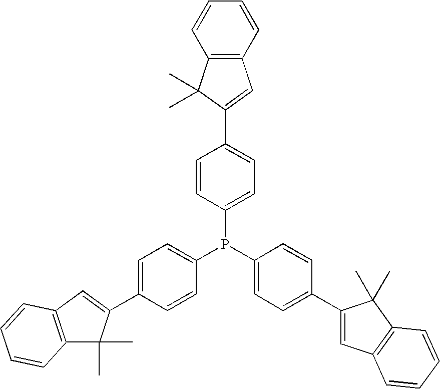

(2)

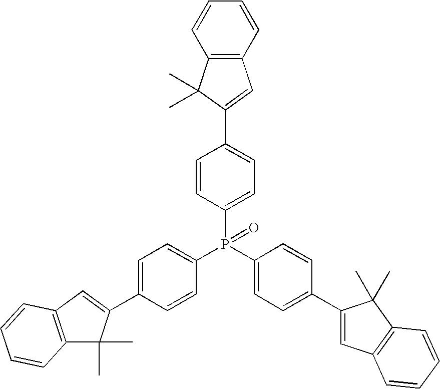

(3)

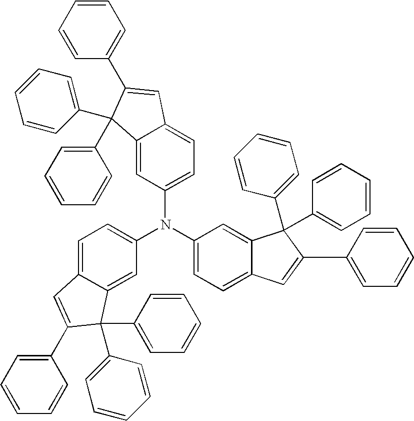

(4)

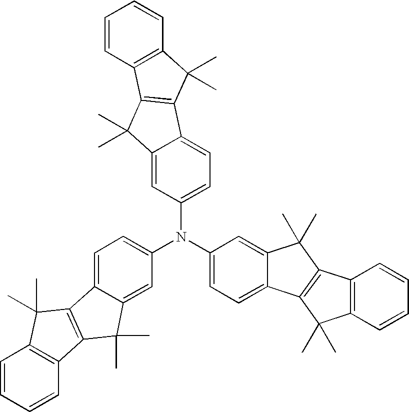

(5)

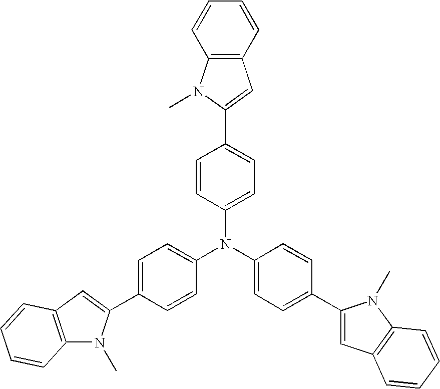

(6)

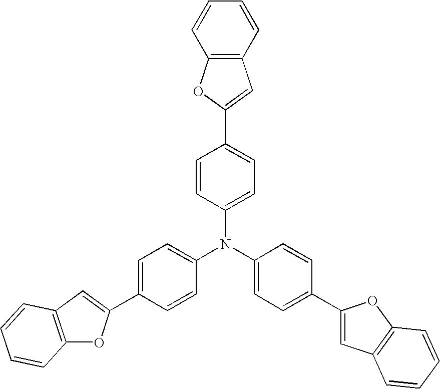

(7)

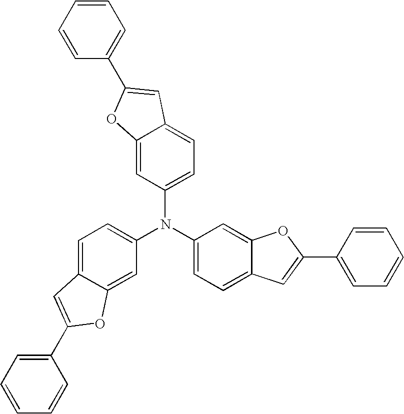

(8)

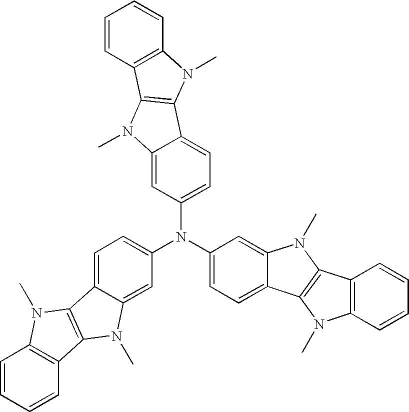

(9)

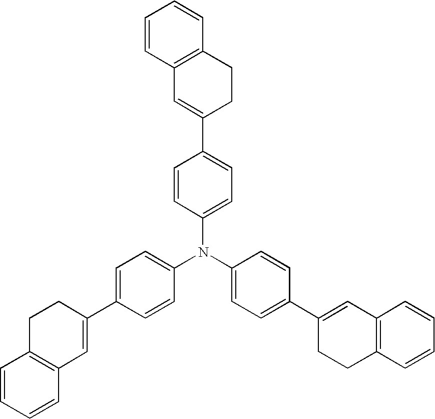

(10)

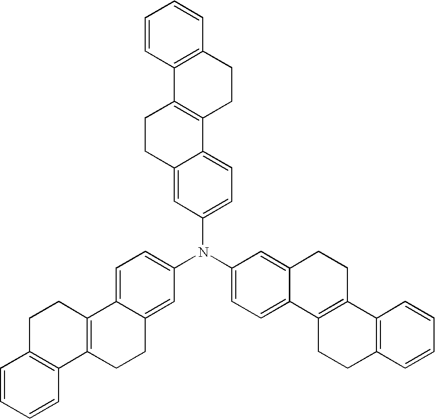

(11)

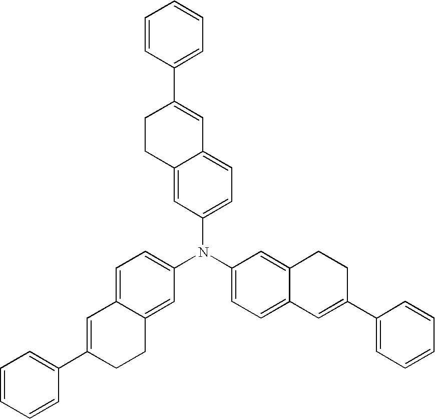

(12)

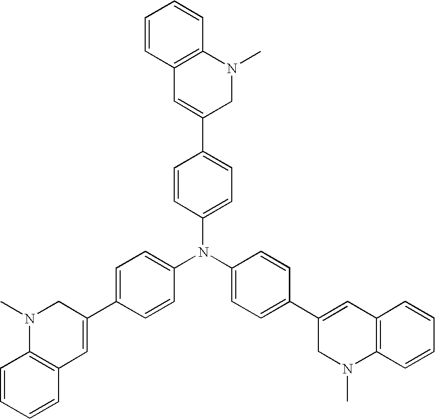

(13)

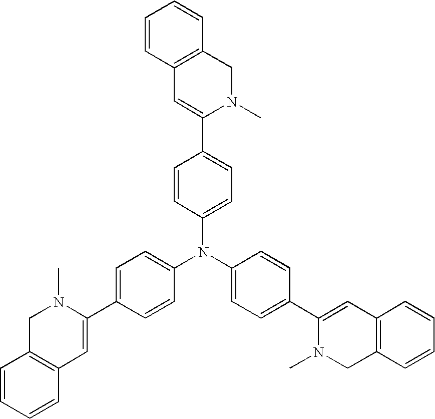

(14)

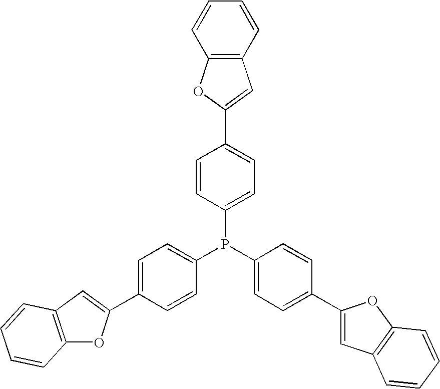

(15)

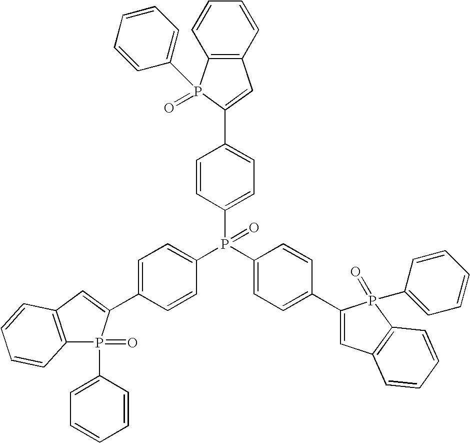

(16)

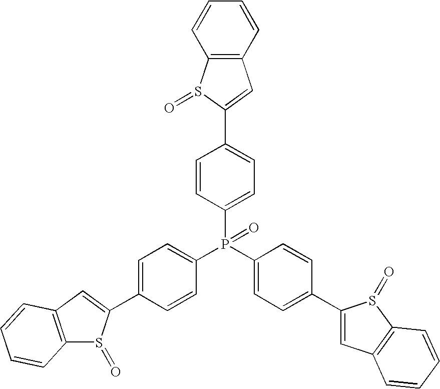

(17)

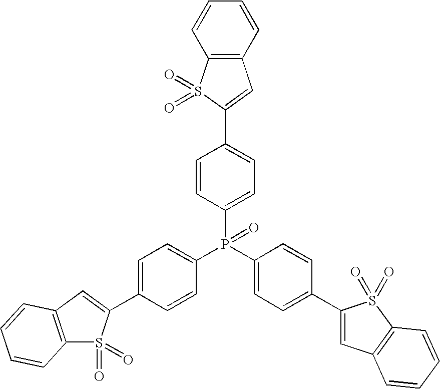

(18)

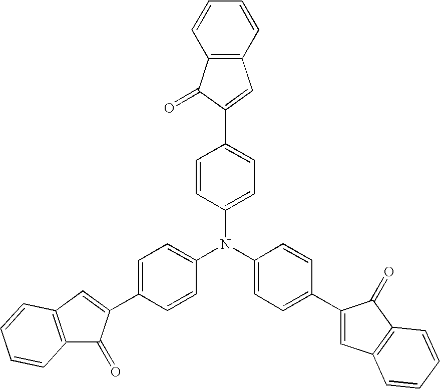

(19)

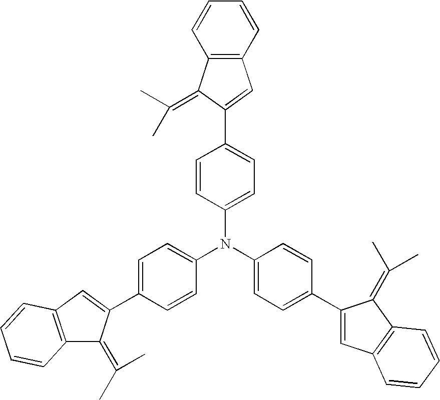

(20)

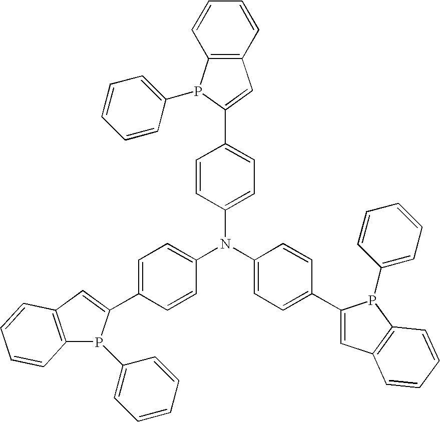

(21)

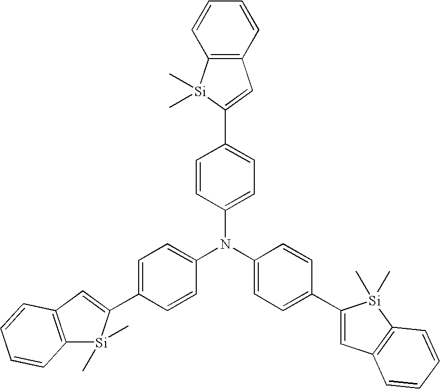

(22)

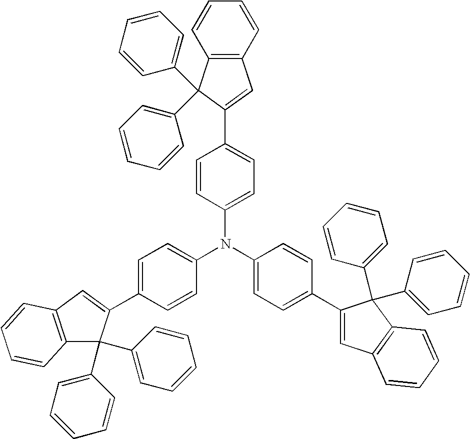

(23)

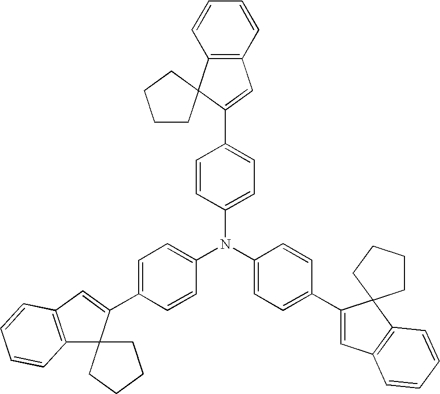

(24)

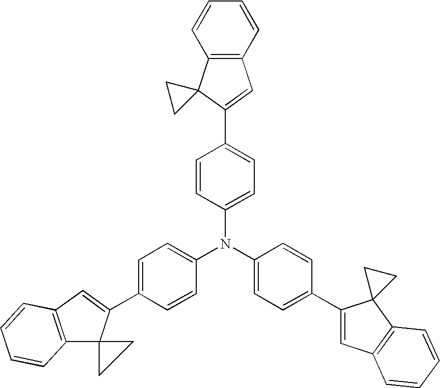

(25)

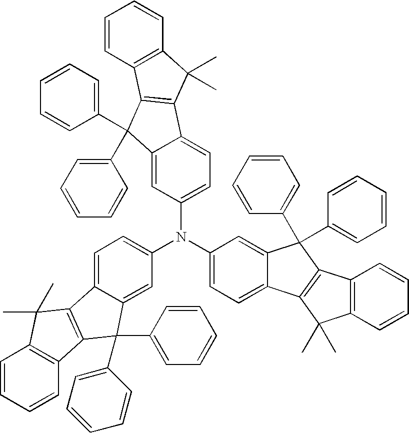

(26)

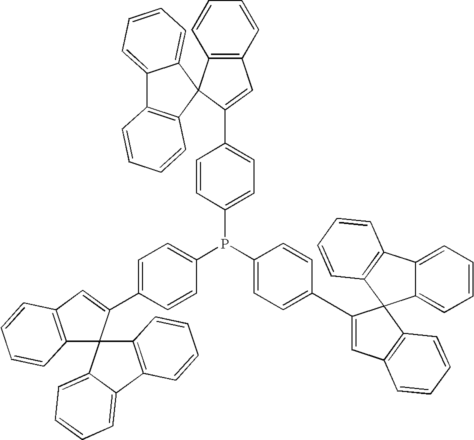

(27)

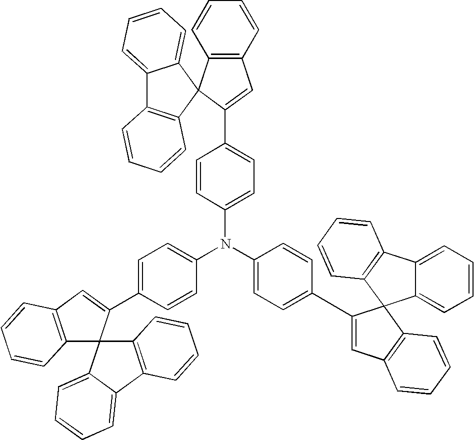

(28)

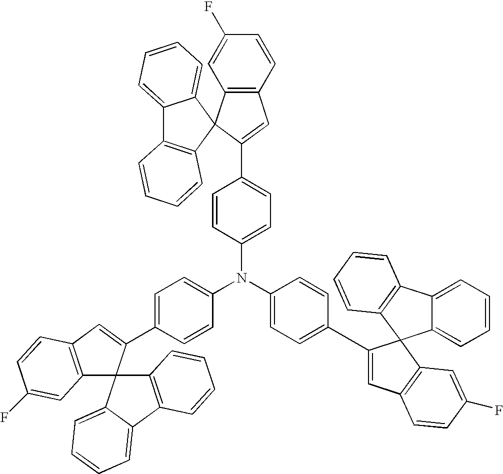

(29)

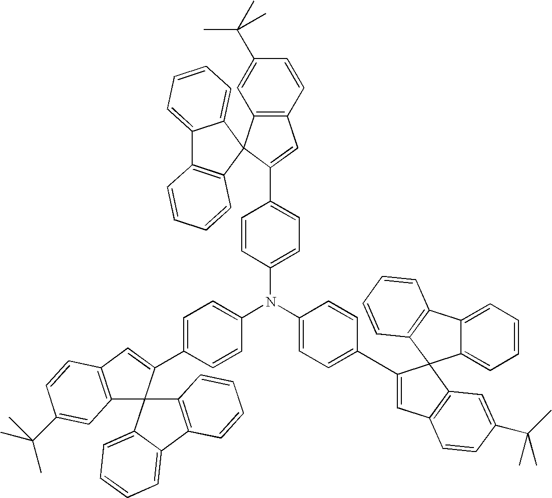

(30)

A very particularly preferred embodiment of the invention is that of compounds of the formulae (4), (5), (6), (7), (8), (9), (10), (11) and (12)

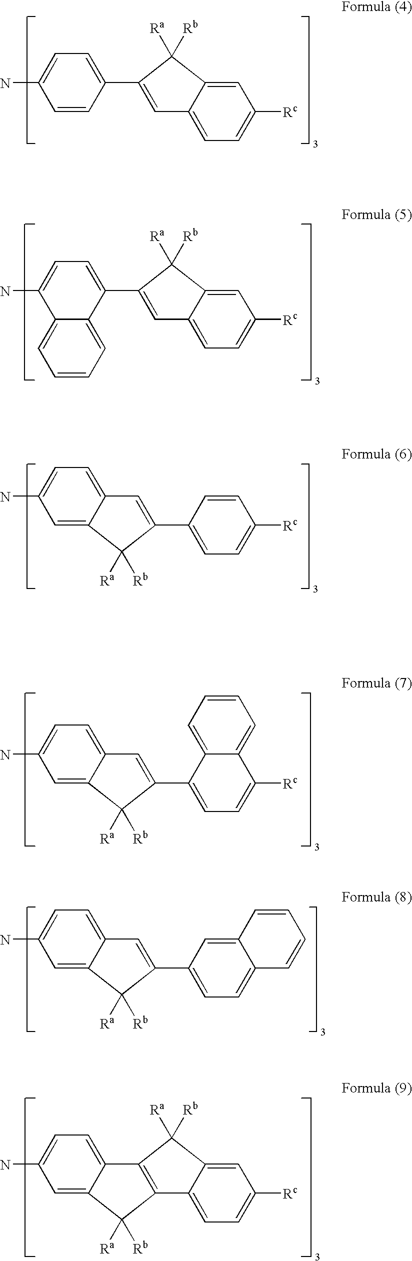

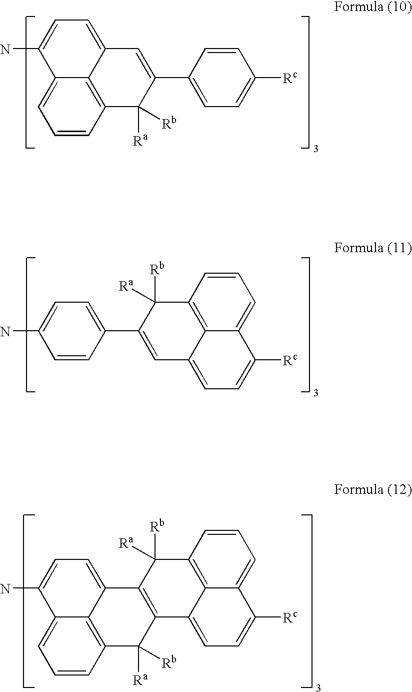

In these compounds, the substituents R^{a}, R^{b} and R^{c} are preferably selected from the substituents as listed in Table 1. These preferred substituent combinations apply to all of the compounds of the formula (4) to formula (12).

TABLE 1
Particularly preferred substituents on the compounds of the formula (4) to
formula (12)

| No. | R^{a} | R^{b} | R^{c} |
|---|---|---|---|
| 1 | H | H | H |
| 2 | H | H | F |
| 3 | H | H | methyl |
| 4 | H | H | tert-butyl |
| 5 | H | H | Br |
| 6 | H | H | phenyl |
| 7 | H | H | para-tolyl |
| 8 | H | H | para-xylyl |
| 9 | H | methyl | H |
| 10 | H | methyl | F |
| 11 | H | methyl | methyl |
| 12 | H | methyl | tert-butyl |
| 13 | H | methyl | Br |
| 14 | H | methyl | phenyl |
| 15 | H | methyl | para-tolyl |
| 16 | H | methyl | para-xylyl |
| 17 | H | F | H |
| 18 | H | F | F |
| 19 | H | F | methyl |
| 20 | H | F | tert-butyl |
| 21 | H | F | Br |
| 22 | H | F | phenyl |
| 23 | H | F | para-tolyl |
| 24 | H | F | para-xylyl |
| 25 | H | CF₃ | H |
| 26 | H | CF₃ | F |
| 27 | H | CF₃ | methyl |
| 28 | H | CF₃ | tert-butyl |
| 29 | H | CF₃ | Br |
| 30 | H | CF₃ | phenyl |
| 31 | H | CF₃ | para-tolyl |
| 32 | H | CF₃ | para-xylyl |
| 33 | H | tert-butyl | H |
| 34 | H | tert-butyl | F |
| 35 | H | tert-butyl | methyl |
| 36 | H | tert-butyl | tert-butyl |
| 37 | H | tert-butyl | Br |
| 38 | H | tert-butyl | phenyl |
| 39 | H | tert-butyl | para-tolyl |
| 40 | H | tert-butyl | para-xylyl |
| 41 | H | phenyl | H |
| 42 | H | phenyl | F |
| 43 | H | phenyl | methyl |
| 44 | H | phenyl | tert-butyl |
| 45 | H | phenyl | Br |
| 46 | H | phenyl | phenyl |
| 47 | H | phenyl | para-tolyl |
| 48 | H | phenyl | para-xylyl |
| 49 | H | para-tolyl | H |
| 50 | H | para-tolyl | F |
| 51 | H | para-tolyl | methyl |
| 52 | H | para-tolyl | tert-butyl |
| 53 | H | para-tolyl | Br |
| 54 | H | para-tolyl | phenyl |
| 55 | H | para-tolyl | para-tolyl |
| 56 | H | para-tolyl | para-xylyl |
| 57 | H | para-fluorophenyl | H |
| 58 | H | para-fluorophenyl | F |
| 59 | H | para-fluorophenyl | methyl |
| 60 | H | para-fluorophenyl | tert-butyl |
| 61 | H | para-fluorophenyl | Br |
| 62 | H | para-fluorophenyl | phenyl |
| 63 | H | para-fluorophenyl | para-tolyl |
| 64 | H | para-fluorophenyl | para-xylyl |
| 65 | H | para-xylyl | H |
| 66 | H | para-xylyl | F |
| 67 | H | para-xylyl | methyl |
| 68 | H | para-xylyl | tert-butyl |
| 69 | H | para-xylyl | Br |
| 70 | H | para-xylyl | phenyl |
| 71 | H | para-xylyl | para-tolyl |
| 72 | H | para-xylyl | para-xylyl |
| 73 | methyl | methyl | H |
| 74 | methyl | methyl | F |
| 75 | methyl | methyl | methyl |
| 76 | methyl | methyl | tert-butyl |
| 77 | methyl | methyl | Br |
| 78 | methyl | methyl | phenyl |
| 79 | methyl | methyl | para-tolyl |
| 80 | methyl | methyl | para-xylyl |
| 81 | methyl | F | H |
| 82 | methyl | F | F |
| 83 | methyl | F | methyl |
| 84 | methyl | F | tert-butyl |
| 85 | methyl | F | Br |
| 86 | methyl | F | phenyl |
| 87 | methyl | F | para-tolyl |
| 88 | methyl | F | para-xylyl |
| 89 | methyl | CF₃ | H |
| 90 | methyl | CF₃ | F |
| 91 | methyl | CF₃ | methyl |
| 92 | methyl | CF₃ | tert-butyl |
| 93 | methyl | CF₃ | Br |
| 94 | methyl | CF₃ | phenyl |
| 95 | methyl | CF₃ | para-tolyl |
| 96 | methyl | CF₃ | para-xylyl |
| 97 | methyl | tert-butyl | H |
| 98 | methyl | tert-butyl | F |
| 99 | methyl | tert-butyl | methyl |
| 100 | methyl | tert-butyl | tert-butyl |
| 101 | methyl | tert-butyl | Br |
| 102 | methyl | tert-butyl | phenyl |
| 103 | methyl | tert-butyl | para-tolyl |
| 104 | methyl | tert-butyl | para-xylyl |
| 105 | methyl | phenyl | H |
| 106 | methyl | phenyl | F |
| 107 | methyl | phenyl | methyl |
| 108 | methyl | phenyl | tert-butyl |
| 109 | methyl | phenyl | Br |
| 110 | methyl | phenyl | phenyl |
| 111 | methyl | phenyl | para-tolyl |
| 112 | methyl | phenyl | para-xylyl |
| 113 | methyl | para-tolyl | H |
| 114 | methyl | para-tolyl | F |
| 115 | methyl | para-tolyl | methyl |
| 116 | methyl | para-tolyl | tert-butyl |
| 117 | methyl | para-tolyl | Br |
| 118 | methyl | para-tolyl | phenyl |
| 119 | methyl | para-tolyl | para-tolyl |
| 120 | methyl | para-tolyl | para-xylyl |
| 121 | methyl | para-fluorophenyl | H |
| 122 | methyl | para-fluorophenyl | F |
| 123 | methyl | para-fluorophenyl | methyl |
| 124 | methyl | para-fluorophenyl | tert-butyl |
| 125 | methyl | para-fluorophenyl | Br |
| 126 | methyl | para-fluorophenyl | phenyl |
| 127 | methyl | para-fluorophenyl | para-tolyl |
| 128 | methyl | para-fluorophenyl | para-xylyl |
| 129 | methyl | para-xylyl | H |
| 130 | methyl | para-xylyl | F |
| 131 | methyl | para-xylyl | methyl |
| 132 | methyl | para-xylyl | tert-butyl |
| 133 | methyl | para-xylyl | Br |
| 134 | methyl | para-xylyl | phenyl |
| 135 | methyl | para-xylyl | para-tolyl |
| 136 | methyl | para-xylyl | para-xylyl |
| 137 | F | F | H |
| 138 | F | F | F |
| 139 | F | F | methyl |
| 140 | F | F | tert-butyl |
| 141 | F | F | Br |
| 142 | F | F | phenyl |
| 143 | F | F | para-tolyl |
| 144 | F | F | para-xylyl |
| 145 | F | CF₃ | H |
| 146 | F | CF₃ | F |
| 147 | F | CF₃ | methyl |
| 148 | F | CF₃ | tert-butyl |
| 149 | F | CF₃ | Br |
| 150 | F | CF₃ | phenyl |
| 151 | F | CF₃ | para-tolyl |
| 152 | F | CF₃ | para-xylyl |
| 153 | F | tert-butyl | H |
| 154 | F | tert-butyl | F |
| 155 | F | tert-butyl | methyl |
| 156 | F | tert-butyl | tert-butyl |
| 157 | F | tert-butyl | Br |
| 158 | F | tert-butyl | phenyl |
| 159 | F | tert-butyl | para-tolyl |
| 160 | F | tert-butyl | para-xylyl |
| 161 | F | phenyl | H |
| 162 | F | phenyl | F |
| 163 | F | phenyl | methyl |
| 164 | F | phenyl | tert-butyl |
| 165 | F | phenyl | Br |
| 166 | F | phenyl | phenyl |
| 167 | F | phenyl | para-tolyl |
| 168 | F | phenyl | para-xylyl |
| 169 | F | para-tolyl | H |
| 170 | F | para-tolyl | F |
| 171 | F | para-tolyl | methyl |
| 172 | F | para-tolyl | tert-butyl |
| 173 | F | para-tolyl | Br |
| 174 | F | para-tolyl | phenyl |
| 175 | F | para-tolyl | para-tolyl |
| 176 | F | para-tolyl | para-xylyl |
| 177 | F | para-fluorophenyl | H |
| 178 | F | para-fluorophenyl | F |
| 179 | F | para-fluorophenyl | methyl |
| 180 | F | para-fluorophenyl | tert-butyl |
| 181 | F | para-fluorophenyl | Br |
| 182 | F | para-fluorophenyl | phenyl |
| 183 | F | para-fluorophenyl | para-tolyl |
| 184 | F | para-fluorophenyl | para-xylyl |
| 185 | F | para-xylyl | H |
| 186 | F | para-xylyl | F |
| 187 | F | para-xylyl | methyl |
| 188 | F | para-xylyl | tert-butyl |
| 189 | F | para-xylyl | Br |
| 190 | F | para-xylyl | phenyl |
| 191 | F | para-xylyl | para-tolyl |
| 192 | F | para-xylyl | para-xylyl |
| 193 | CF₃ | CF₃ | H |
| 194 | CF₃ | CF₃ | F |
| 195 | CF₃ | CF₃ | methyl |
| 196 | CF₃ | CF₃ | tert-butyl |
| 197 | CF₃ | CF₃ | Br |
| 198 | CF₃ | CF₃ | phenyl |
| 199 | CF₃ | CF₃ | para-tolyl |
| 200 | CF₃ | CF₃ | para-xylyl |
| 201 | CF₃ | tert-butyl | H |
| 202 | CF₃ | tert-butyl | F |
| 203 | CF₃ | tert-butyl | methyl |
| 204 | CF₃ | tert-butyl | tert-butyl |
| 205 | CF₃ | tert-butyl | Br |
| 206 | CF₃ | tert-butyl | phenyl |
| 207 | CF₃ | tert-butyl | para-tolyl |
| 208 | CF₃ | tert-butyl | para-xylyl |
| 209 | CF₃ | phenyl | H |
| 210 | CF₃ | phenyl | F |
| 211 | CF₃ | phenyl | methyl |
| 212 | CF₃ | phenyl | tert-butyl |
| 213 | CF₃ | phenyl | Br |
| 214 | CF₃ | phenyl | phenyl |
| 215 | CF₃ | phenyl | para-tolyl |
| 216 | CF₃ | phenyl | para-xylyl |
| 217 | CF₃ | para-tolyl | H |
| 218 | CF₃ | para-tolyl | F |
| 219 | CF₃ | para-tolyl | methyl |
| 220 | CF₃ | para-tolyl | tert-butyl |
| 221 | CF₃ | para-tolyl | Br |
| 222 | CF₃ | para-tolyl | phenyl |
| 223 | CF₃ | para-tolyl | para-tolyl |
| 224 | CF₃ | para-tolyl | para-xylyl |
| 225 | CF₃ | para-fluorophenyl | H |
| 226 | CF₃ | para-fluorophenyl | F |
| 227 | CF₃ | para-fluorophenyl | methyl |
| 228 | CF₃ | para-fluorophenyl | tert-butyl |
| 229 | CF₃ | para-fluorophenyl | Br |
| 230 | CF₃ | para-fluorophenyl | phenyl |
| 231 | CF₃ | para-fluorophenyl | para-tolyl |
| 232 | CF₃ | para-fluorophenyl | para-xylyl |
| 233 | CF₃ | para-xylyl | H |
| 234 | CF₃ | para-xylyl | F |
| 235 | CF₃ | para-xylyl | methyl |
| 236 | CF₃ | para-xylyl | tert-butyl |
| 237 | CF₃ | para-xylyl | Br |
| 238 | CF₃ | para-xylyl | phenyl |
| 239 | CF₃ | para-xylyl | para-tolyl |
| 240 | CF₃ | para-xylyl | para-xylyl |
| 241 | tert-butyl | tert-butyl | H |
| 242 | tert-butyl | tert-butyl | F |
| 243 | tert-butyl | tert-butyl | methyl |
| 244 | tert-butyl | tert-butyl | tert-butyl |
| 245 | tert-butyl | tert-butyl | Br |
| 246 | tert-butyl | tert-butyl | phenyl |
| 247 | tert-butyl | tert-butyl | para-tolyl |
| 248 | tert-butyl | tert-butyl | para-xylyl |
| 249 | tert-butyl | phenyl | H |
| 250 | tert-butyl | phenyl | F |
| 251 | tert-butyl | phenyl | methyl |
| 252 | tert-butyl | phenyl | tert-butyl |
| 253 | tert-butyl | phenyl | Br |
| 254 | tert-butyl | phenyl | phenyl |
| 255 | tert-butyl | phenyl | para-tolyl |
| 256 | tert-butyl | phenyl | para-xylyl |
| 257 | tert-butyl | para-tolyl | H |
| 258 | tert-butyl | para-tolyl | F |
| 259 | tert-butyl | para-tolyl | methyl |
| 260 | tert-butyl | para-tolyl | tert-butyl |
| 261 | tert-butyl | para-tolyl | Br |
| 262 | tert-butyl | para-tolyl | phenyl |
| 263 | tert-butyl | para-tolyl | para-tolyl |
| 264 | tert-butyl | para-tolyl | para-xylyl |
| 265 | tert-butyl | para-fluorophenyl | H |
| 266 | tert-butyl | para-fluorophenyl | F |
| 267 | tert-butyl | para-fluorophenyl | methyl |
| 268 | tert-butyl | para-fluorophenyl | tert-butyl |
| 269 | tert-butyl | para-fluorophenyl | Br |
| 270 | tert-butyl | para-fluorophenyl | phenyl |
| 271 | tert-butyl | para-fluorophenyl | para-tolyl |
| 272 | tert-butyl | para-fluorophenyl | para-xylyl |
| 273 | tert-butyl | para-xylyl | H |
| 274 | tert-butyl | para-xylyl | F |
| 275 | tert-butyl | para-xylyl | methyl |
| 276 | tert-butyl | para-xylyl | tert-butyl |
| 277 | tert-butyl | para-xylyl | Br |
| 278 | tert-butyl | para-xylyl | phenyl |
| 279 | tert-butyl | para-xylyl | para-tolyl |
| 280 | tert-butyl | para-xylyl | para-xylyl |
| 281 | phenyl | phenyl | H |
| 282 | phenyl | phenyl | F |
| 283 | phenyl | phenyl | methyl |
| 284 | phenyl | phenyl | tert-butyl |
| 285 | phenyl | phenyl | Br |
| 286 | phenyl | phenyl | phenyl |
| 287 | phenyl | phenyl | para-tolyl |
| 288 | phenyl | phenyl | para-xylyl |
| 289 | phenyl | para-tolyl | H |
| 290 | phenyl | para-tolyl | F |
| 291 | phenyl | para-tolyl | methyl |
| 292 | phenyl | para-tolyl | tert-butyl |
| 293 | phenyl | para-tolyl | Br |
| 294 | phenyl | para-tolyl | phenyl |
| 295 | phenyl | para-tolyl | para-tolyl |
| 296 | phenyl | para-tolyl | para-xylyl |
| 297 | phenyl | para-fluorophenyl | H |
| 298 | phenyl | para-fluorophenyl | F |
| 299 | phenyl | para-fluorophenyl | methyl |
| 300 | phenyl | para-fluorophenyl | tert-butyl |
| 301 | phenyl | para-fluorophenyl | Br |
| 302 | phenyl | para-fluorophenyl | phenyl |
| 303 | phenyl | para-fluorophenyl | para-tolyl |
| 304 | phenyl | para-fluorophenyl | para-xylyl |
| 305 | phenyl | para-xylyl | H |
| 306 | phenyl | para-xylyl | F |
| 307 | phenyl | para-xylyl | methyl |
| 308 | phenyl | para-xylyl | tert-butyl |
| 309 | phenyl | para-xylyl | Br |
| 310 | phenyl | para-xylyl | phenyl |
| 311 | phenyl | para-xylyl | para-tolyl |
| 312 | phenyl | para-xylyl | para-xylyl |
| 313 | para-tolyl | para-tolyl | H |
| 314 | para-tolyl | para-tolyl | F |
| 315 | para-tolyl | para-tolyl | methyl |
| 316 | para-tolyl | para-tolyl | tert-butyl |
| 317 | para-tolyl | para-tolyl | Br |
| 318 | para-tolyl | para-tolyl | phenyl |
| 319 | para-tolyl | para-tolyl | para-tolyl |
| 320 | para-tolyl | para-tolyl | para-xylyl |
| 321 | para-tolyl | para-fluorophenyl | H |
| 322 | para-tolyl | para-fluorophenyl | F |
| 323 | para-tolyl | para-fluorophenyl | methyl |
| 324 | para-tolyl | para-fluorophenyl | tert-butyl |
| 325 | para-tolyl | para-fluorophenyl | Br |
| 326 | para-tolyl | para-fluorophenyl | phenyl |
| 327 | para-tolyl | para-fluorophenyl | para-tolyl |
| 328 | para-tolyl | para-fluorophenyl | para-xylyl |
| 329 | para-tolyl | para-xylyl | H |
| 330 | para-tolyl | para-xylyl | F |
| 331 | para-tolyl | para-xylyl | methyl |
| 332 | para-tolyl | para-xylyl | tert-butyl |
| 333 | para-tolyl | para-xylyl | Br |
| 334 | para-tolyl | para-xylyl | phenyl |
| 335 | para-tolyl | para-xylyl | para-tolyl |
| 336 | para-tolyl | para-xylyl | para-xylyl |
| 337 | para-fluorophenyl | para-fluorophenyl | H |
| 338 | para-fluorophenyl | para-fluorophenyl | F |
| 339 | para-fluorophenyl | para-fluorophenyl | methyl |
| 340 | para-fluorophenyl | para-fluorophenyl | tert-butyl |
| 341 | para-fluorophenyl | para-fluorophenyl | Br |
| 342 | para-fluorophenyl | para-fluorophenyl | phenyl |
| 343 | para-fluorophenyl | para-fluorophenyl | para-tolyl |
| 344 | para-fluorophenyl | para-fluorophenyl | para-xylyl |
| 345 | para-fluorophenyl | para-xylyl | H |
| 346 | para-fluorophenyl | para-xylyl | F |
| 347 | para-fluorophenyl | para-xylyl | methyl |
| 348 | para-fluorophenyl | para-xylyl | tert-butyl |
| 349 | para-fluorophenyl | para-xylyl | Br |
| 350 | para-fluorophenyl | para-xylyl | phenyl |
| 351 | para-fluorophenyl | para-xylyl | para-tolyl |
| 352 | para-fluorophenyl | para-xylyl | para-xylyl |
| 353 | para-xylyl | para-xylyl | H |
| 354 | para-xylyl | para-xylyl | F |
| 355 | para-xylyl | para-xylyl | methyl |
| 356 | para-xylyl | para-xylyl | tert-butyl |
| 357 | para-xylyl | para-xylyl | Br |
| 358 | para-xylyl | para-xylyl | phenyl |
| 359 | para-xylyl | para-xylyl | para-tolyl |
| 360 | para-xylyl | para-xylyl | para-xylyl |

| | | |
|---|---|---|
| 361 | 2,2′-biphenyl | H |
| 362 | 2,2′-biphenyl | F |
| 363 | 2,2′-biphenyl | methyl |
| 364 | 2,2′-biphenyl | tert-butyl |
| 365 | 2,2′-biphenyl | Br |
| 366 | 2,2′-biphenyl | phenyl |
| 367 | 2,2′-biphenyl | para-tolyl |
| 368 | 2,2′-biphenyl | para-xylyl |

The invention further provides for the use of compounds of the formula (1) or formula (3) in organic electronic devices, in particular in organic electroluminescent devices.

The invention further provides organic electroluminescent devices comprising cathode, anode and at least one emitting layer, characterized in that at least one organic layer comprises at least one compound of the formula (1) or formula (3).

Apart from the emitting layer, the organic electroluminescent device may comprise further layers. These may, for example, be: hole injection layer, hole transport layer, hole blocking layer, electron transport layer and/or electron injection layer. However, it should be pointed out here that not necessarily each of these layers has to be present. For instance, especially when compounds of the formula (1) or formula (3) are used as a dopant with electron-conducting host materials, very good results are is still obtained when the organic electroluminescent device does not contain any separate electron transport layer and the emitting layer directly adjoins the electron injection layer or the cathode. It may likewise be preferred when the organic electroluminescent device does not contain any separate hole transport layer and the emitting layer directly adjoins the hole injection layer or the anode. It may further be preferred when the compound of the formula (1) or formula (3) is used not only as the dopant in the emitting layer, but also additionally as a hole-conducting compound (as a pure substance or as a mixture) in the hole transport layer.

The compound of the formula (1) or formula (3) can perform different functions in the organic electroluminescent device. These depend upon the precise structure of this compound. Especially the selection of the A and Z groups determines the particularly suitable function of these compounds. For instance, these compounds can be used in particular as emitters, as hole transport materials, as electron transport materials, or, in electrophosphorescent devices, also as matrix materials or as hole blocking materials.

Suitable emitters are in particular compounds in which the symbols A and Z are:
A is N at each instance;
Z is the same or different at each instance and is C(R¹)₂, C(R¹)₂—C(R¹)₂ or C(R¹)₂—NR¹.

Suitable hole transport materials are in particular compounds in which the symbols A and Z are:
A is N or P at each instance;
Z is the same or different at each instance and is C(R¹)₂, C(R¹)₂—C(R¹)₂, C(R¹)₂—NR¹, O, NR¹ or PR¹, in particular O, NR¹ or PR¹.

Suitable electron transport materials for fluorescent or phosphorescent devices are in particular compounds in which the symbols A and Z are
A is P═O at each instance;
Z is the same or different at each instance and is C(R¹)₂, C(R¹)₂—C(R¹)₂, C(R¹)₂—NR¹, C═O, S═O, SO₂, BR¹ or PR¹O, in particular C═O, S═O, SO₂, BR¹ or PR¹O.

Suitable matrix materials and hole blocking materials for electrophosphorescent devices are in particular compounds in which the symbols A and Z are:
A is P═O at each instance;
Z is the same or different at each instance and is C(R¹)₂, C(R¹)₂—C(R¹)₂, C(R¹)₂—NR¹, C═O, S═O, SO₂ or PR¹O, in particular C═O, S═O, SO₂ or PR¹O.

When the compound of the formula (1) or formula (3) is used as an emitter, it is preferably used together with a host material. The proportion of the compound of the formula (1) or formula (3) in the mixture is then between 0.1 and 99% by weight, preferably between 0.5 and 50% by weight, more preferably between 1 and 20% by weight, in particular between 1 and 10% by weight. Correspondingly, the proportion of the host material in the mixture is between 1 and 99.9% by weight, preferably between 50 and 99.5% by weight, more preferably between 80 and 99% by weight, in particular between 90 and 99% by weight.

Preference is further given to organic electroluminescent devices, characterized in that a plurality of emitting compounds are used in the same layer or in different layers, of which at least one of these compounds has a structure of the formula (1). More preferably, these compounds together have a plurality of emission maxima between 380 nm and 750 nm, so that white emission results overall, i.e. apart from the compound of the formula (1) at least one further emitting compound which fluoresces or phosphoresces and which emits yellow, orange or red light is also used.

Preferred host materials are organic compounds, whose emission is at a shorter wavelength than that of the compound of the formula (1) or which do not emit at all in the visible region. Useful host materials are various substance classes. Preferred host materials are selected from the classes of the oligoarylenes (for example 2, 2′,7,7′-tetraphenylspirobifluorene according to EP 676461 or dinaphthylanthracene), of the atropisomers (for example according to the unpublished application EP 04026402.0, of the oligoarylenevinylenes (for example DPVBi or spiro-DPVBi according to EP 676461), of the polypodal metal complexes (for example according to WO 04/081017), of the hole-conducting compounds (for example according to WO 04/058911) or of the electron-conducting compounds, in particular ketones, phosphine oxides and sulphoxides (for example according to the unpublished patent application DE 102004008304.5). Particularly preferred host materials are selected from the classes of the oligoarylenes, including naphthalene, anthracene and/or pyrene, of the oligoarylenevinylenes, of the ketones, of the phosphine oxides and of the sulphoxides.

When the compound of the formula (1) or formula (3) is used, as a hole transport material, electron transport material or as a hole blocking material, it is preferred when this compound is used as the pure substance. Especially as a hole transport material and as an electron transport material, these compounds are also suitable for use in further organic electronic devices, for example in organic transistors.

When the compound of the formula (1) or formula (3) is used as a matrix material for electrophosphorescent devices, its proportion in the mixture is between 1 and 99.9% by weight, preferably between 30 and 99.5% by weight, more preferably between 50 and 99% by weight, in particular between 80 and 99% by weight. Correspondingly, the proportion of the emitter which emits light from the triplet state and therefore exhibits electrophosphorescence in the mixture is between 0.1 and 99% by weight, preferably between 0.5 and 70% by weight, more preferably between 1 and 50% by weight, in particular between 1 and 20% by weight.

The mixing ratios can be adjusted by mixing in solvents (or solvent mixtures) or by co-evaporation under reduced pressure, in a carrier gas stream or under vacuum.

Preference is further given to an organic electroluminescent device, characterized in that one or more layers are applied by a sublimation process. In this process, the materials are applied by vapour deposition in vacuum sublimation units at a pressure of less than 10⁻⁵ mbar, preferably less than 10⁻⁶ mbar, more preferably less than 10⁻⁷ mbar.

Preference is likewise given to an organic electroluminescent device, characterized in that one or more layers are applied by the OVPD (Organic Vapour Phase Deposition) process or with the aid of carrier gas sublimation. In this process, the materials are applied at a pressure between 10⁻⁵ mbar and 1 bar.

Preference is further given to an organic electroluminescent device, characterized in that one or more layers are produced from solution, for example by spin-coating, or by any printing process, for example screenprinting, flexographic printing or offset printing, but more preferably LITI (Light Induced Thermal Imaging, thermal transfer printing) or inkjet printing.

The above-described compounds in organic electronic devices have the following surprising advantages over the prior art:
1. The efficiency of corresponding devices is higher in comparison to systems according to the prior art. We suspect that this comes about as a result of the suppressed cis-trans isomerization about the double bond.
2. The stability of corresponding devices is better in comparison to systems according to the prior art, which is shown in particular in a higher lifetime. This higher stability possibly also comes about as a result of the hindered cis-trans isomerization, since it is known that cis-stilbene systems, which can form when isomerization is possible, react further photochemically to give dihydrophenanthrene systems and then, by oxidative subsequent reaction, to give phenanthrene systems. This side reaction is not possible in the inventive systems.
3. The compounds can be sublimed and applied by vapour deposition without marked decomposition, are thus easier to purify and to process, and are therefore better suited to use in OLEDs than materials according to the prior art, especially than materials which comprise stilbene units which are not substituted on the double bond.
4. Since the inventive compounds do not undergo any isomerization in the preparation and processing and in the operation of the electronic device, the reproducibility of the device is increased.

The present application text and also the examples which follow below are aimed at the use of inventive compounds in relation to OLEDs and the corresponding displays. In spite of this restriction of the description, it is possible for those skilled in the art without any further inventive activity also to utilize the inventive compounds for further uses in other electronic devices, for example for organic field-effect transistors (O-FETs), organic thin-film transistors (O-TFTs), organic integrated circuits (O-ICs), organic solar cells (O-SCs), organic light-emitting transistors (O-LETs), organic field-quench devices (O-FQDs), light-emitting electrochemical cells (LECs) or else organic laser diodes (O-Laser), to name just a few applications. The use of the inventive compounds in the corresponding devices, just like these devices themselves, likewise form part of the subject-matter of the present invention. The invention is also given here in detail by the examples which follow without any intention to restrict it thereto

### EXAMPLES

The syntheses which follow were, unless stated otherwise, carried out under a protective gas atmosphere. The reactants were purchased from Aldrich. Tris(p-bromophenyl)amine was synthesized according to J. Am. Chem. Soc. 1987, 109, 4960-4968. 2-Indenylboronic acid was synthesized according to J. Org. Chem. 2002, 67, 169-176; 2-bromobenzyl diethylphosphonate was synthesized according to Z. Anorg. Allg. Chem. 1994, 620(12), 2041-7.

### Example 1

### Synthesis of 4,4′,4″ Tris(1H-indene-2-yl)triphenylamine (amine 1)

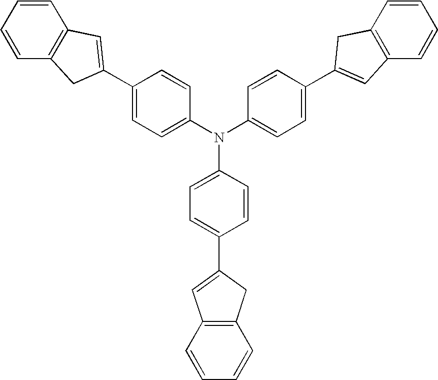

800 mg (0.72 mmol) of Pd(PPh₃)₄ are added to a nitrogen-saturated mixture of 15.0 g (31 mmol) of tris(p-bromophenyl)amine, 19.8 g (124 mmol) or 2-indenylboronic acid, 40.0 g (188 mmol) of K₃PO₄, 1 l of dioxane and 1 l of water. The suspension is heated to 80° C. for 7 h. Afterwards, 0.09 g of NaCN is added and the aqueous phase is removed. The organic phase is washed twice with H₂O and subsequently dried over Na₂SO₄. After the removal of the solvents and repeated recrystallization from toluene, yellow needles are obtained which, by HPLC, have a purity of approximately 99.9%. The yield is 16 g (88%).

¹H NMR (CDCl₃, 500 MHz): δ [ppm]=3.78 (s, 6H), 7.13-7.19 (m, 12H), 7.23-7.28 (m, 3H), 7.38 (d, J=7.4 Hz, 3H), 7.46 (d, J=7.4 Hz, 3H), 7.56 (d, J=8.7 Hz, 6H).

### Example 2

### Synthesis of 4,4′,4″tris(1,1,3-trimethyl-1H-inden-2-yl)triphenylamine (amine 2)

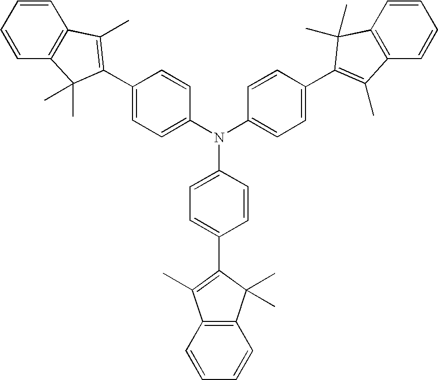

4.8 g (200 mmol) of sodium hydride are added in portions to a mixture of 33.2 g (55 mmol) of 4,4′,4″-tris(1H-inden-2-yl)triphenylamine and 300 ml of dimethylformamide. Subsequently, the suspension is heated to 80° C., admixed dropwise at this temperature with 18.7 ml (300 mmol) of methyl iodide and subsequently stirred at 80° C. for a further 50 h. Afterwards, the mixture is admixed at room temperature with 100 ml of ammonia solution and 500 ml of ethyl acetate, and the aqueous phase is removed. The organic phase is washed 5 times with 300 ml of water and subsequently dried over Na₂SO₄. After the removal of the solvent and repeated recrystallization from toluene (1.5 ml/g), yellow needles are obtained which, by HPLC, have a purity of 99.8%. The yield is 23.7 g (60.3%). The sublimation was effected at a pressure of approx. 5×10⁻⁵ mbar and T=300 C.

¹H NMR (CDCl₃, 500 MHz): δ [ppm]=1.51 (s, 18H), 2.10 (s, 9H), 7.14-7.20 (m, 9H), 7.24-7.27 (m, 6H), 7.40 (d, 3H), 7.47 (d, 3H), 7.58 (m, 3H).

### Example 3

### Synthesis of 4,4′,4″-tris(spiro(fluorene-9,1′ inden-2-yl)triphenylamine (amine 3)

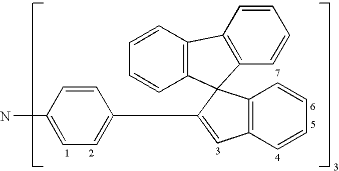

### a) Tris(4-(2-bromophenylvinyl)phenyl)amine

A mixture, cooled to 0° C., of 107.5 g (350 mmol) of 2-bromobenzyl diethylphosphonate and 1000 ml of DMF is admixed with 67.3 g (700 mmol) of sodium tert-butoxide and stirred for 30 min. This mixture is admixed with a solution of 29.6 g (90 mmol) of tris(4-formylphenyl)amine in 1000 ml of DMF at 0° C. over 30 min. and subsequently stirred at 0 C for a further 4 h. This mixture is then admixed with stirring with 250 ml of 2.5 N aqueous hydrochloric acid, subsequently with 600 ml of water and then with 200 ml of ethanol, and stirred for a further 30 min. The precipitate is filtered off with suction, washed twice with 200 ml each time of a mixture of water/ethanol (1:1, v:v) and three times with 200 ml each time of ethanol, and subsequently dried under reduced pressure. The solid thus obtained is recrystallized from 160 ml of DMF, finally extracted from 500 ml of hot ethanol by stirring and dried under reduced pressure.

The yield at a purity of 99.0% by ¹H NMR is 51.0 g (65 mmol) corresponding to 71.8% of theory.

¹H NMR (CDCl₃, 500 MHz): δ [ppm]=7.66 (d, 3H), 7.58 (d, 3H), 7.47 (d, 6H), 7.39 (d, 3H), 7.30 (dd, 3H), 7.13 (d, 6H), 7.10 (dd, 3H), 7.01 (d, 3H).

### b) 4,4′,4″-Tris(spiro(fluorene-9,1′-inden-2-yl)triphenylamine

An efficiently stirred suspension of 7.9 g (10 mmol) of tris(4-(2-bromophenylvinyl)phenyl)amine in 300 ml of diethyl ether is admixed at −78° C. dropwise with 14.4 ml (36 mmol) of n-butyllithium (2.5 molar in n-hexane). After stirring for a further 15 min., the cold bath is removed and the reaction mixture is allowed to warm to room temperature and subsequently stirred at room temperature for a further 3 h. The suspension is then admixed dropwise at room temperature with a solution of 7.2 g (40 mmol) of fluorenone in 200 ml of diethyl ether, and stirred at room temperature for a further 14 h. The yellow suspension is admixed with a mixture of 10 ml of acetic acid and 200 ml of water and stirred thoroughly for 30 min. The organic phase is removed, washed twice with 500 ml of water and concentrated to dryness. The solid is taken up in 500 ml of toluene, admixed with 1.0 g of p-toluenesulphonic acid and boiled on a water separator until no further water separates out (approx. 3 h). After cooling, the yellow solution is filtered through silica gel, the toluene is removed under reduced pressure, and the residue is recrystallized three times from DMF (10 ml/g) and five times from dioxane (10 ml/g).

The yield at a purity of 99.9% by ¹H NMR is 5.1 g (4.9 mmol), corresponding to 49.2% of theory. The sublimation was effected at a pressure of approx. 5×10⁻⁵ mbar and T=380° C.

¹H NMR (TCE-d2+1 μl of hydrazine hydrate, 500 MHz): δ [ppm]=7.81 (d, 6H, fluorene), 7.42 (s, 3H, H3), 7.40 (d, 3H, H4), 7.32 (dd, 6H, fluorene), 7.17 (dd, 3H, H5), 7.07 (dd, 6H, fluorene), 6.87 (dd, 3H, H6), 6.81 (d, 6H, fluorene), 6.76 (d, 6H, H2), 6.43 (d, 6H, H1), 6.38 (d, 3H, H7).

### c) Investigation of the Redox Stability

4,4′,4″-Tris(spiro(fluorene-9,1′-inden-2-yl)triphenylamine is oxidized in solution under air to the corresponding radical cation, recognizable by a high line broadening of the ¹H NMR signals of the phenyl-indenyl part of the spectrum. The reduction to the amine can be effected, for example, with hydrazine hydrate (see ¹H NMR spectrum). This redox reaction is reversible; no decomposition of the molecule can be observed even on repeated performance of the redox cycle.

### Example 4

### Synthesis of 4,4′,4′-tris(1,1′-di-para-tolylinden-2-yl)triphenylamine (amine 4)

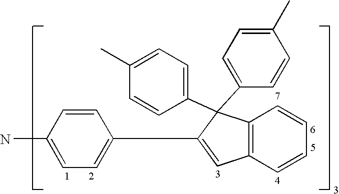

### a) 4,4′,4″-Tris(1,1′-di-para-tolylinden-2-yl)triphenylamine

An efficiently stirred suspension of 7.9 g (10 mmol) of tris(4-(2-bromophenylvinyl)-phenyl)amine in 300 ml of diethyl ether is admixed dropwise at −78° C. with 14.4 ml (36 mmol) of n-butyllithium (2.5 molar in n-hexane). After stirring for a further 15 min., the cold bath is removed, and the reaction mixture is allowed to warm to room temperature and subsequently stirred at room temperature for a further 3 h. The suspension is admixed dropwise at room temperature with a solution of 8.4 g (40 mmol) of 4,4′-dimethylbenzophenone in 200 ml of diethyl ether and stirred at room temperature for a further 14 h. The yellow suspension is admixed with a mixture of 10 ml of acetic acid and 200 ml of water and stirred thoroughly for 30 ml The organic phase is removed, washed twice with 500 ml of water and concentrated to dryness. The solid is taken up in 500 ml of toluene, admixed with 1.0 g of p-toluenesulphonic acid and boiled on a water separator until no further water separates out (approx. 3 h). After cooling, the yellow solution is filtered through silica gel, the toluene is removed under reduced pressure and the residue is recrystallized three times from DM F (10 ml/g) and five times from dioxane (8 ml/g).

The yield at a purity of 99.9% by ¹H NMR is 5.9 g (5.2 mmol), corresponding to 51.4% of theory. The sublimation was effected at a pressure of approx. 5×10⁻⁵ mbar and T=370 C.

¹H NMR (TCE-d2+1 μl of hydrazine hydrate, 500 MHz): δ [ppm]=7.40 (s, 3H, H3), 7.38 (d, 3H, H4), 7.20 (m, 12H, AB-p-tolyl), 7.19 (dd, 3H, H5), 6.89 (dd, 3H, H6), 6.76 (d, 6H, H2), 6.62 (m, 12H, AB-p-tolyl), 6.37 (d, 6H, H1), 6.36 (d, 3H, H7), 2.41 (s, 18H, CH₃).

### b) Investigation of the Redox Stability

4,4′,4″-Tris(1,1′-di-para-tolyl-inden-2-yl)triphenylamine is oxidized in solution under air to the corresponding radical cation, recognizable by a high line broadening of the ¹H NMR signals of the phenyl-indenyl part of the spectrum. The reduction to the amine can be effected, for example, with hydrazine hydrate (see ¹H NMR spectrum). This redox reaction is reversible; no decomposition of the molecule is observed, even on repeated performance of the redox cycles.

### Example 5

### Synthesis of 4,4′,4″-tris[5,5,10,10-di-spiro-fluorenyl-5,10-dihydroindeno[2,1-a]inden-2-yl]amine (amine 5)

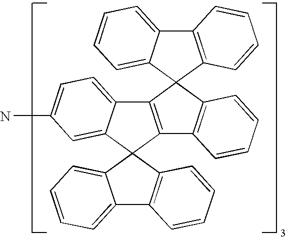

### a) 4,4′,4″-Tris[2,3-dibromo-spiro(fluorene-9,1′-inden-2-yl)]triphenylamine

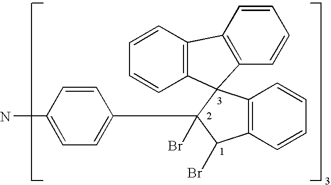

A mixture, cooled to 0° C., of 51.9 g (50 mmol) of 4,4′,4″-tris(spiro(fluorene-91′-inden-2-yl)triphenylamine (synthesized according to Example 3) dissolved in 1000 ml of dichloromethane is admixed with exclusion of light at 0° C. with a mixture of 2.6 ml (50 mmol) of bromine and 100 ml of dichloromethane, and stirred at 0° C. for 4 h. After the dichloromethane has been removed under reduced pressure, the residue is washed with a little ethanol, recrystallized once from toluene and dried under reduced pressure. The yield, at a purity of about 97.0% by HPLC, is 69.4 g (46 mmol), corresponding to 91.5% of theory.

### b) 4,4′,4″-Tris[3-bromo-spiro(fluorene-1′,9-1H-inden-2-yl)]triphenylamine

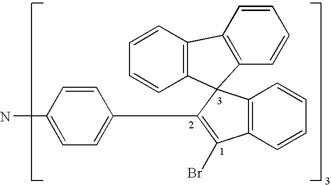

An efficiently stirred suspension of 40.0 g (26 mmol) of 4,4′,4″-tris[2,3-dibromo-spiro(fluorene-9,1′-inden-2-yl)]triphenylamine in 300 ml THF is admixed in portions at 0 C with 5.1 g (45 mmol) of potassium tert-butoxide and stirred at room temperature for 1 h. Subsequently, the mixture is poured into 100 ml of ice-water and extracted three times with 200 ml each time of dichloromethane, and the dichloromethane phase is washed three times with 300 ml of water, and once with 300 ml of saturated NaCl solution, and then dried over sodium sulphate. After the solvent has been removed under reduced pressure, the residue is recrystallized from DMF. The yield, at a purity of about 98.0% by HPLC, is 28.7 g (22.5 mmol) corresponding to 85.4% of theory.

### c) 4,4′,4″-Tris[5,5,10,10-di-spiro-fluorenyl-5,10-dihydroindeno[2,1-a]inden-2-yl]amine

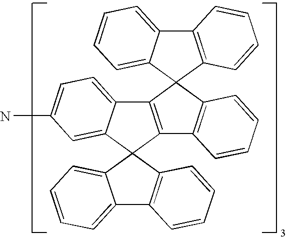

An efficiently stirred suspension of 12.8 g (10 mmol) of 4,4′,4″-tris[3-bromo-spiro(fluorene-1′,9-1H-inden-2-yl)]triphenylamine in 300 ml of diethyl ether is admixed dropwise at −78° C. with 14.4 ml (36 mmol) of n-butyllithium (2.5 molar in n-hexane). After stirring for a further 15 min., the cold bath is removed, and the reaction mixture is allowed to warm to room temperature and subsequently stirred at room temperature for a further 3 h. The suspension is then admixed dropwise at room temperature with a solution of 7.9 g (44 mmol) of fluorenone in 200 ml of diethyl ether, and stirred at room temperature for a further 14 h. The yellow suspension is admixed with a mixture of 10 ml of acetic acid and 200 ml of water, and stirred thoroughly for 30 min. The organic phase is removed, washed twice with 500 ml of water and concentrated to dryness. The solid is taken up in 500 ml of toluene, admixed with 500 mg of p-toluenesulphonic acid and boiled on a water separator until no further water separates out (approx. 3 h). After cooling, the yellow solution is filtered through silica gel, the toluene is removed under reduced pressure and the residue is recrystallized three times from DMF (15 ml/g) and five times from dioxane (12 ml/g). The yield at a purity of 99.9% by ¹H NMR is 7.5 g (4.9 mmol) corresponding to 49.2% of theory. The sublimation was effected at a pressure of approx. 5×10⁻⁵ mbar and T=395° C.

¹H NMR (TCE-d2+1 μl of hydrazine hydrate, 500 MHz): δ [ppm]=7.81 (d, 6H, fluorene), 7.77 (d, 6H, fluorene), 7.40 (d, 3H), 7.32 (dd, 6H, fluorene), 7.30 (dd, 6H, fluorene), 7.17 (dd, 3H), 7.09 (d, 3H), 7.07 (dd, 6H, fluorene), 7.01 (dd, 6H, fluorene), 6.87 (dd, 3H), 6.85 (d, 6H, fluorene), 6.80 (d, 6H, fluorene), 6.66 (dd, 3H), 6.46 (dd, 3H), 6.38 (d, 3H), 6.28 (d, 3H).

### c) Investigation of the Redox Stability

4,4′,4″-Tris[5,5,10,10-di-spiro-fluorenyl-5,10-dihydroindeno[2,1-a]inden-2-yl]amine is oxidized in solution under air to the corresponding radical cation, recognizable by a high line broadening of the ¹H NMR signals of the phenyl-indenyl part of the spectrum. The reduction to the amine can be effected, for example, with hydrazine hydrate (see ¹H NMR spectrum). This redox reaction is reversible; no decomposition of the molecule is observed, even on repeated performance of the redox cycle.

### Example 6

### Production of the OLEDs

OLEDs are produced by a general process according to WO 04/058911, which is adapted in the individual case to the particular circumstances (for example layer thickness variation in order to achieve optimal efficiency and colour).

In the Examples 7 to 11 which follow, the results of various OLEDs are presented. The fundamental structure, the materials and layer thicknesses used, apart from the emitting layer and electron transport layer, are identical in Examples 7 to 10 for better comparability. Analogous to the abovementioned general process, OLEDs with the following structure are obtained (except that the hole transport layer detailed below is not relevant for Example 7):

| | |
|---|---|
| Loch injection | 20 nm of PEDOT (spin-coated from water; purchased |
| layer (HIL) | from H. C. Starck, Goslar, Germany; |
| | poly(3,4-ethylenedioxy-2,5-thiophene)) |
| Hole transport | 20 nm of NaphDATA (applied by vapour deposition; |
| layer (HTM) | purchased from SynTec, Wolfen, Germany; 4,4′,4″- |
| | tris(N-1-naphthyl-N-phenylamino)triphenylamine) |
| Hole transport | 20 nm of S-TAD (applied by vapour deposition; |
| layer (HTM) | prepared according to WO 99/12888; 2,2′,7,7′- |
| | tetrakis(diphenylamino)-spiro-9,9′-bifluorene) |
| Emission layer | See Table 1 for materials, concentrations and layer |
| (EML) | thicknesses |
| Electron | See Table 1 for materials and layer thickness |
| conductor (ETL) | |
| Ba—Al (cathode) | 3 nm of Ba, 150 nm of Al thereon. |

In Example 7 the hole transport materials used, apart from the two mentioned above, are NPB (N-naphthyl-N-phenyl-4,4′-diaminobiphenyl) and HTM1 (2,2′,7,7′-tetrakis(di-para-tolylamino)spiro-9,9′-bifluorene).

These OLEDs which are yet to be optimized are characterized in a standard manner; for this purpose, the electroluminescence spectra, the efficiency (measured in cd/A), the power efficiency (measured in Im/W) as a function of brightness, calculated from current-voltage-brightness characteristics (IUL characteristics), and the lifetime are determined. The lifetime is defined as the time after which the starting brightness of the OLED has fallen by half at a constant current density of 10 mAcm².

Table 1 then summarizes the results of some OLEDs (Examples 7 to 10) with the composition of the EML and the ETL including the layer thicknesses also being listed in each case. The emitting layers comprise, as emitting materials of the formula (1), the dopant D1 (according to structure Example 1). The comparative examples used are OLEDs which comprise, as emitting compounds, the dopant D2 according to the abovementioned prior art or only the host material. The host materials used are the compounds H1 to H4 depicted below. The electron transport materials used are ETM1 (AlQ₃, purchased from SynTec, tris(quinolinato)aluminium(III)) or E™2 bis(9,9′-spirobifluoren-2-yl)phenylphosphine oxide according to WO 05/003253). Table 2 summarizes the results of further OLEDs (Example 11) which comprises, as emitting materials of the formula (1), the dopant D1 or the dopant D3 and which have been optimized for better efficiency and lifetime by variation of the hole transport layers.

For better clarity, the corresponding structural formulae of the dopants and host materials used are shown below:

TABLE 1

| | |
|---|---|
| | Dopant D1 |
| 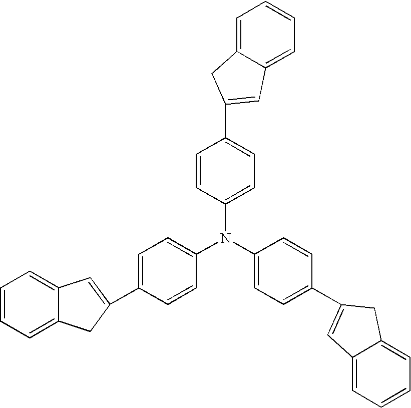 | |
| | Dopant D2 |
| 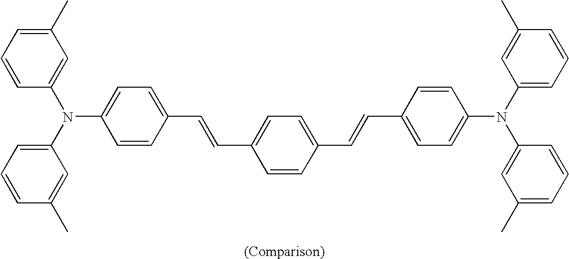 | |
| | Dopant D3 |
| 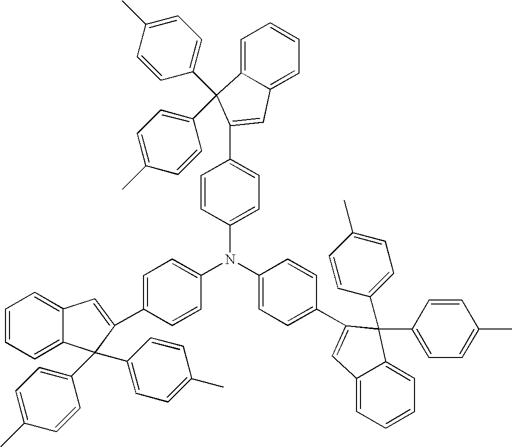 | |

| | |
|---|---|
| | Host H1 |
| 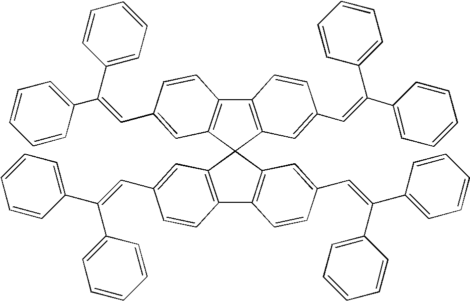 | |
| | Host H2 |
| 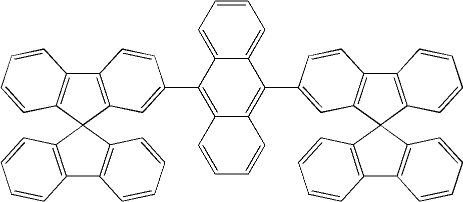 | |
| | Host H3 |
| 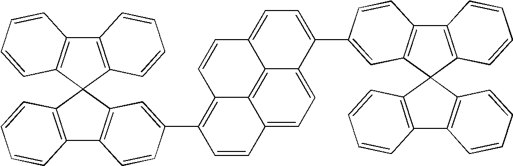 | |
| | Host H4 |
| 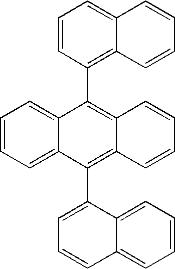 | |

| | | | Max. Efficiency | Voltage (V) at | | |
|---|---|---|---|---|---|---|
| Example | EML | ETL | (cd/A) | 100 cd/m² | CIE | Lifetime (h) |
| Example 7a | H1 | ETM1 | 4.2 | 5.8 | x = 0.17; y = 0.26 | 1200 |
| (Comparison) | (30 nm) | (20 nm) | | | | |
| Example 7b | H1:D2 (5%) | ETM1 | 4.9 | 6.3 | x = 0.17; y = 0.31 | 1000 |
| (Comparison) | (30 nm) | (20 nm) | | | | |
| Example 7c | H1:D1 (2%) | ETM1 | 4.0 | 4.8 | x = 0.18; y = 0.26 | 3100 |
| | (30 nm) | (20 nm) | | | | |
| Example 7d | H1:D1 (5%) | ETM1 | 4.2 | 4.7 | x = 0.19; y = 0.28 | 2500 |
| | (30 nm) | (20 nm) | | | | |
| Example 7e | H1:D1 (5%) | ETM2 | 4.0 | 4.6 | x = 0.18; y = 0.26 | 3600 |
| | (30 nm) | (20 nm) | | | | |
| Example 7f | H1:D1 (5%) | ETM2 | 4.2 | 4.7 | x = 0.16; y = 0.25 | 3900 |
| | (30 nm) | (30 nm) | | | | |
| Example 8a | H2 | ETM1 | 3.3 | 6.5 | x = 0.15; y = 0.15 | 700 |
| (Comparison) | (30 nm) | (20 nm) | | | | |
| Example 8b | H2:D1 (2%) | ETM1 | 3.9 | 5.2 | x = 0.16; y = 0.21 | 2500 |
| | (30 nm) | (20 nm) | | | | |
| Example 9a | H3 | ETM1 | 3.7 | 5.8 | x = 0.17; y = 0.28 | 1400 |
| (Comparison) | (30 nm) | (20 nm) | | | | |
| Example 9b | H3:D2 (5%) | ETM1 | 5.4 | 6.5 | x = 0.19; y = 0.37 | 1000 |
| (Comparison) | (30 nm) | (20 nm) | | | | |
| Example 9c | H3:D1 (2%) | ETM1 | 3.2 | 5.5 | x = 0.17; y = 0.18 | 2500 |
| | (30 nm) | (20 nm) | | | | |
| Example 9d | HS:D1 (5%) | ETM1 | 3.5 | 5.3 | x = 0.19; y = 0.22 | 4100 |
| | (30 nm) | (20 nm) | | | | |
| Example 10a | H4 | ETM1 | 1.1 | 5.8 | x = 0.17; y = 0.19 | 3100 |
| (Comparison) | (30 nm) | (20 nm) | | | | |
| Example 10b | H4:D1 (1%) | ETM1 | 3.8 | 5.1 | x = 0.15; y = 0.14 | 3000 |
| | (30 nm) | (20 nm) | | | | |
| Example 10c | H4:D1 (2%) | ETM1 | 4.5 | 5.3 | x = 0.15; y = 0.16 | 3500 |
| | (30 nm) | (20 nm) | | | | |
| Example 10d | H4:D1 (5%) | ETM1 | 4.0 | 4.8 | x = 0.16; y = O.18 | 4000 |
| | (30 nm) | (20 nm) | | | | |
| Example 10e | H4:D1 (2%) | ETM2 | 4.7 | 5.2 | x = 0.15; y = 0.15 | 5300 |
| | (30 nm) | (20 nm) | | | | |
| Example 10f | H4:D1 (5%) | ETM2 | 4.3 | 4.6 | x = 0.15; y = 0.17 | 4800 |
| | (30 nm) | (20 nm) | | | | |

TABLE 2

| | | | | | Max. Efficiency | Voltage (V) at | | |
|---|---|---|---|---|---|---|---|---|
| Example | HTL1 | HTL2 | EML | ETL | (cd/A) | 100 cd/m² | CIE | Lifetime (h) |
| Example 11a | HTM1 | S-TAD | H4: D1 (2%) | ETM1 | 4.7 | 4.8 | x = 0.15; y = 0.15 | 5500 |
| | (20 nm) | (20 nm) | (30 nm) | (20 nm) | | | | |
| Example 11b | NaphDATA | NPB | H4: D1 (2%) | ETM1 | 4.9 | 5.0 | x = 0.15; y = 0.15 | 4500 |
| | (20 nm) | (20 nm) | (30 nm) | (20 nm) | | | | |
| Example 11c | HTM1 | NPB | H4: D1 (2%) | ETM1 | 4.3 | 4.9 | x = 0.15; y = 0.15 | 6200 |
| | (20 nm) | (20 nm) | (30 nm) | (20 nm) | | | | |
| Example 12a | HTM1 | S-TAD | H4: D1 (2%) | ETM1 | 3.8 | 4.9 | x = 0.15; y = 0.11 | 4000 |
| | (20 nm) | (20 nm) | (30 nm) | (20 nm) | | | | |
| Example 12b | NaphDATA | NPB | H4: D1 (2%) | ETM1 | 4.0 | 5.1 | x = 0.15; y = 0.11 | 3800 |
| | (20 nm) | (20 nm) | (30 nm) | (20 nm) | | | | |
| Example 12c | HTM1 | NPB | H4: D1 (2%) | ETM1 | 3.5 | 5.2 | x = 0.15; y = 0.11 | 4300 |
| | (20 nm) | (20 nm) | (30 nm) | (20 nm) | | | | |

1-17. (canceled)

18. A compound of the formula (1), 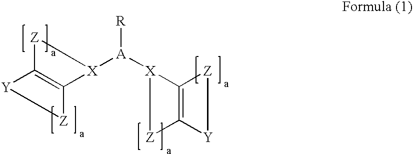
wherein
A is the same or different at each instance and is N, P or P═O;
X and Y are the same or different at each instance and are each an aromatic or heteroaromatic system which has 5 to 60 aromatic ring atoms that is optionally substituted by one or more R¹ radicals;
R is the same or different at each instance and is a straight-chain alkyl group having 1 to 40 carbon atoms or a branched or cyclic alkyl group having 3 to 40 carbon atoms, each of which is optionally substituted by one or more R¹ radicals, in which one or more nonadjacent CH₂ groups is optionally replaced by —R²C═CR²—, —C≡C—, P(═O)(R), SO, SO₂, Si(R²)₂, Ge(R²)₂, Sn(R²)₂, C═O, C═S, C═Se, C═NR², —O—, —S— or —CONR²—, and in which one or more hydrogen atoms is optionally replaced by F, Cl, Br, I, CN or NO₂, or an aromatic or heteroaromatic system which has 5 to 40 aromatic ring atoms and is optionally substituted by one or more R² radicals, or an aryloxy or heteroaryloxy group which has 5 to 40 aromatic ring atoms and is optionally substituted by one or more R² radicals, or a combination of two, three or four of these systems; or R is a group of the formula (2), 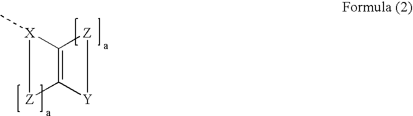
where the symbols are each defined as described above and below, and the dashed bond symbolizes the attachment to A;
Z is the same or different at each instance and is a bivalent group C(R¹)₂, C═O, C[═C(R¹)₂], Si(R¹)₂, O, S═O, SO₂O, NR, BR¹, PR¹, PR¹O, C(R¹)₂—C(R¹)₂, C(R¹)₂—NR¹, C(R¹)₂—C(R¹)₂—C(R¹)₂ or C(R¹)₂—O—C(R¹)₂, where Z, apart from to the double bond, also bonds to the Y group or to the X group on the same double bond and thus forms a further cyclic ring system;
R¹ is the same or different at each instance and is H, F, Cl, Br, I, CN, NO₂, a straight-chain alkyl-, alkoxy- or thioalkoxy group having 1 to 40 carbon atoms or a branched or cyclic alkyl, alkoxy or thioalkoxy group which has 3 to 40 carbon atoms, and is optionally substituted in each case by one or more R² radicals in which one or more nonadjacent CH₂ groups is optionally replaced by —R²C═CR²—, —C≡C—, P(═O)(R²), SO, SO₂, Si(R)₂, Ge(R²)₂, Sn(R²)₂, C═O, C═S, C═Se, C═NR², —O—, —S— or —CONR²— and in which one or more hydrogen atoms is optionally replaced by F, Cl, Br, I, CN or NO₂, or an aromatic or heteroaromatic system which has 5 to 40 aromatic ring atoms and is optionally substituted by one or more R² radicals, or an aryloxy- or heteroaryloxy group which has 5 to 40 aromatic ring atoms and is optionally substituted by one or more R² radicals, or a combination of two, three or four of these systems; in this radical, two or more substituents R¹ together may also form a mono- or polycyclic aliphatic ring system;
R² is the same or different at each instance and is H or an aliphatic or aromatic hydrocarbon radical having 1 to 20 carbon atoms; and
a is the same or different at each instance and is 0 or 1, with the proviso that at least one index is a=1 per double bond, where a 0 means that, instead of Z, an R¹ group is bonded to the double bond and to X or Y.

19. The compound according to claim 18, wherein
A is N or P at each instance;
X is the same or different at each instance and is a bivalent aryl or heteroaryl group which has 5 to 25 aromatic ring atoms, and is optionally substituted by one, two, three or four R¹ radicals;
Y is the same or different at each instance and is a monovalent aryl or heteroaryl group which has 5 to 25 aromatic ring atoms, and is optionally substituted by one, two, three or four R¹ radicals;
R is a group of the abovementioned formula (2) at each instance;
Z is the same or different at each instance and is C(R¹)₂, SO₂, BR¹, P(R¹)O, C(R¹)₂—C(R¹)₂, C(R¹)₂—NR¹;
a is equal to 0 or 1 at each instance, where one index is a 0 and the other index is a=1 on each double bond;
R¹ and R² are each as defined under claim 18.

20. The compound according to claim 18, wherein
A is N at each instance;
X is the same or different at each instance and is a bivalent aryl group which has 6 to 16 carbon atoms, and is optionally substituted by one or two R¹ radicals;
Y is the same or different at each instance and is a monovalent aryl group which has 6 to 16 carbon atoms, and is optionally substituted by one, two or three R¹ radicals;
Z is the same or different at each instance and is C(R¹)₂, P(R¹)O or C(R¹)₂—C(R¹)₂;
R, R¹, R² and a are each as defined under claim 18.

21. The compound according to claim 18, wherein X represent the same aromatic or heteroaromatic system.

22. The compound according to claim 18, wherein X is benzene or naphthalene and are all identically substituted.

23. The compound according to claim 18, wherein Y represents the same aromatic or heteroaromatic system.

24. The compound according to claim 18, wherein Y represents benzene or naphthalene and are all identically substituted.

25. The compound according to claim 18, wherein Z are each selected identically.

26. The compound according to claim 18, wherein they have a symmetrical structure and a threefold rotational axis.

27. The compound according to claim 18, wherein the compound of formula (1) corresponds to the compound of formula (3) 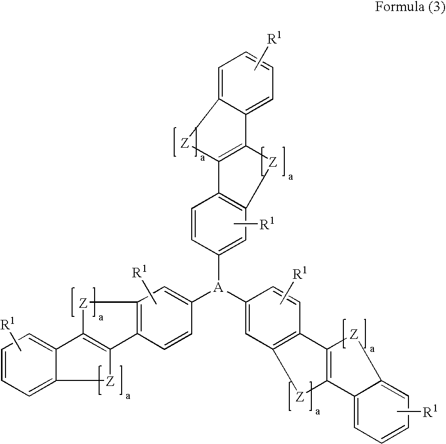
wherein
A is the same or different at each instance and is N, P or P═O;
Z is the same or different at each instance and is a bivalent group C(R¹)₂, C═O, C[═C(R¹)₂], Si(R¹)₂, O, S═O, SO₂, NR, BR¹, PR¹, PR¹O, C(R¹)₂—C(R¹)₂, C(R¹)₂—NR¹, C(R¹)₂—C(R¹)₂—C(R¹)₂ or C(R¹)₂—O—C(R¹)₂, where Z, apart from to the double bond forms a further cyclic ring system;
R¹ is the same or different at each instance and is H, F, Cl, Br, I, CN, NO₂, a straight-chain alkyl-, alkoxy- or thioalkoxy group having 1 to 40 carbon atoms or a branched or cyclic alkyl alkoxy or thioalkoxy group which has 3 to 40 carbon atoms, and is optionally substituted in each case by one or more R² radicals in which one or more nonadjacent CH₂ groups is optionally replaced by —R²C═CR²—, —C≡C—, P(═O)(R²), SO, SO₂, Si(R²)₂, Ge(R²)₂, Sn(R²)₂, C═O, C═S, C═Se, C═NR², —O—, —S— or —CONR²— and in which one or more hydrogen atoms is optionally replaced by F, Cl, Br, I, CN or NO₂, or an aromatic or heteroaromatic system which has 5 to 40 aromatic ring atoms and is optionally substituted by one or more R² radicals, or an aryloxy- or heteroaryloxy group which has 5 to 40 aromatic ring atoms and is optionally substituted by one or more R² radicals, or a combination of two, three or four of these systems; in this radical, two or more substituents R¹ together may also form a mono- or polycyclic aliphatic ring system; and
a is the same or different at each instance and is 0 or 1, with the proviso that at least one index is a=1 per double bond, where a=0 means that, instead of Z, an R¹ group is bonded to the double bond and to X or Y,
where the maximum number of substituents R¹ corresponds to the number of substitutable hydrogen atoms.

28. The compound according to claim 18, wherein the compound of formula (1) is selected from the structures (1) to (30) which is optionally substituted by R¹ or unsubstituted

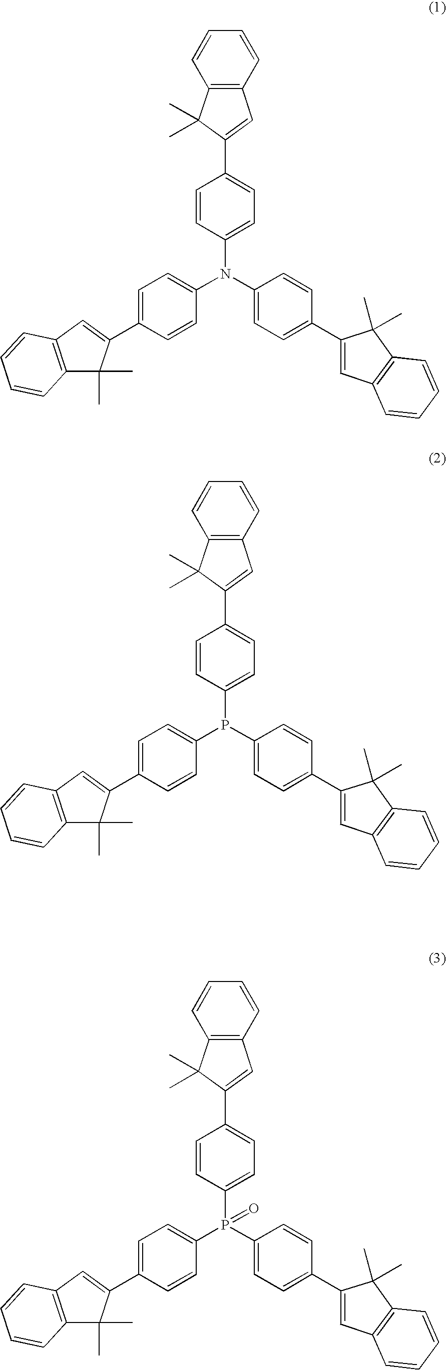

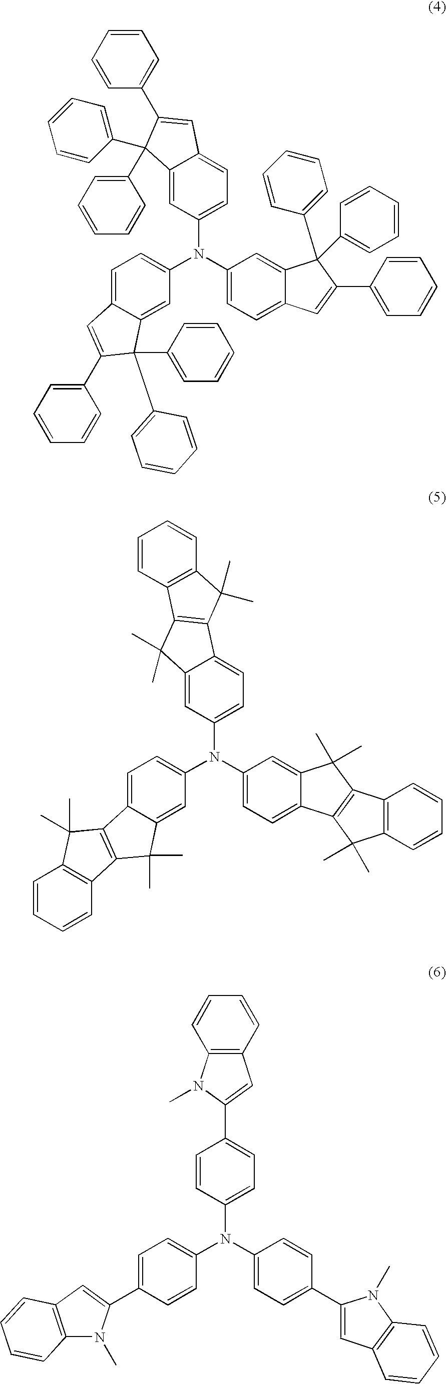

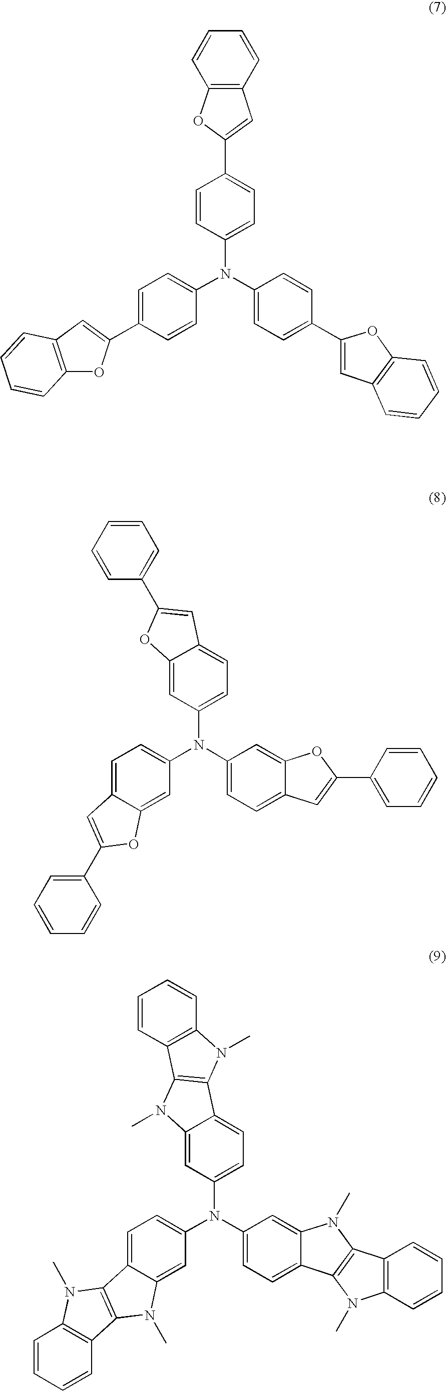

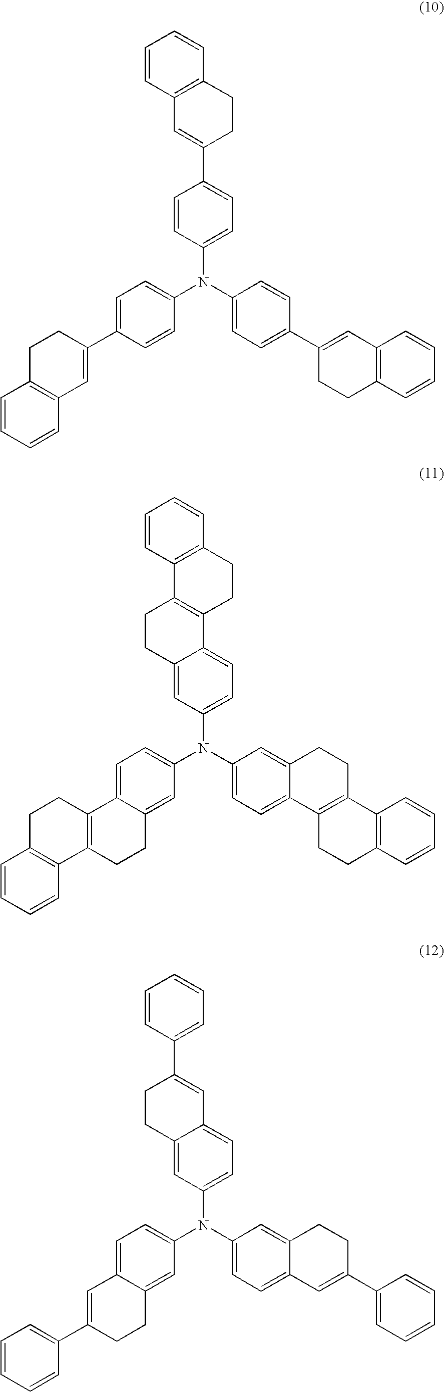

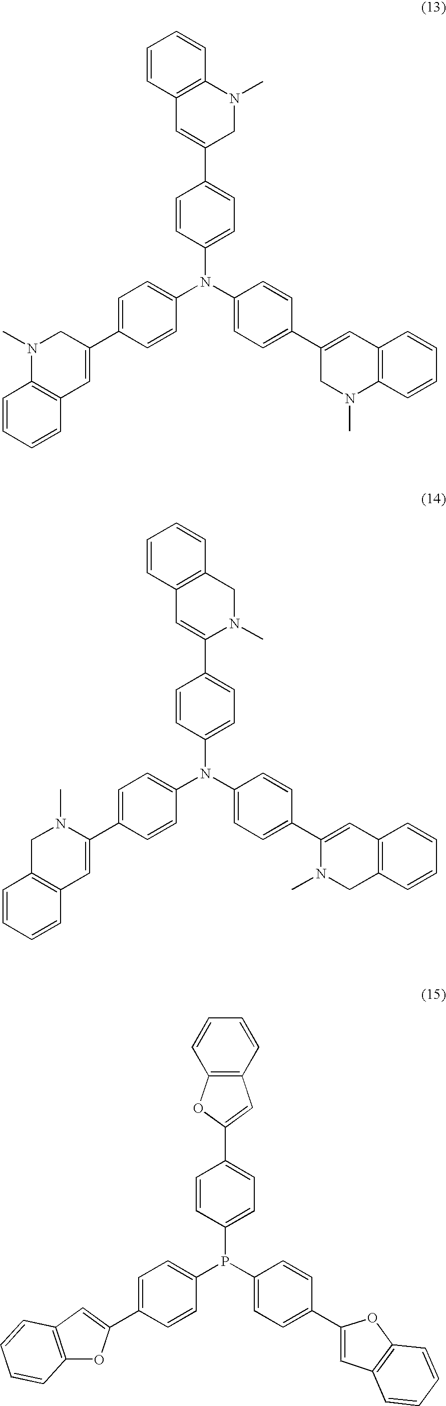

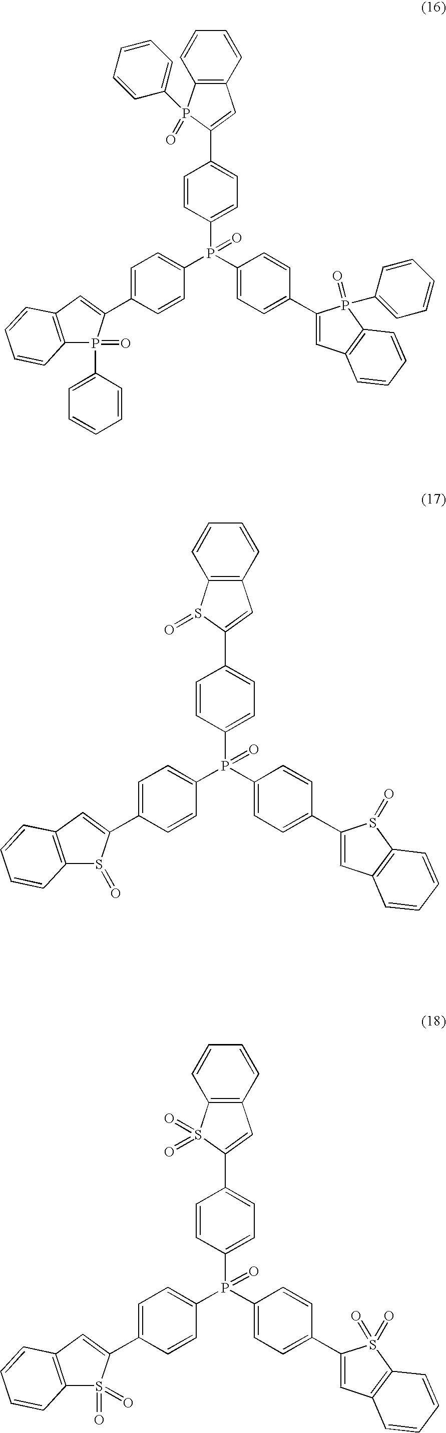

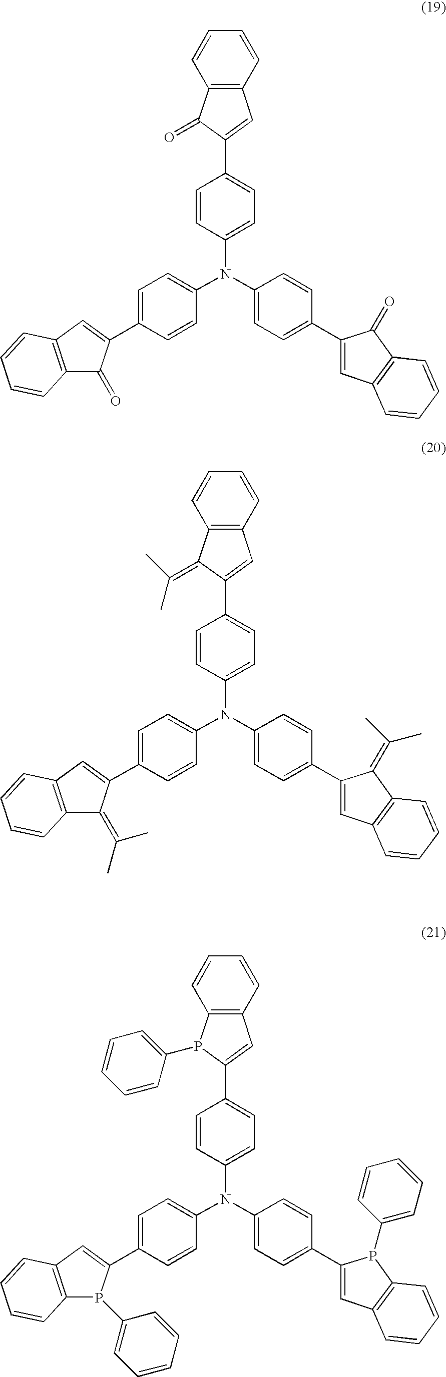

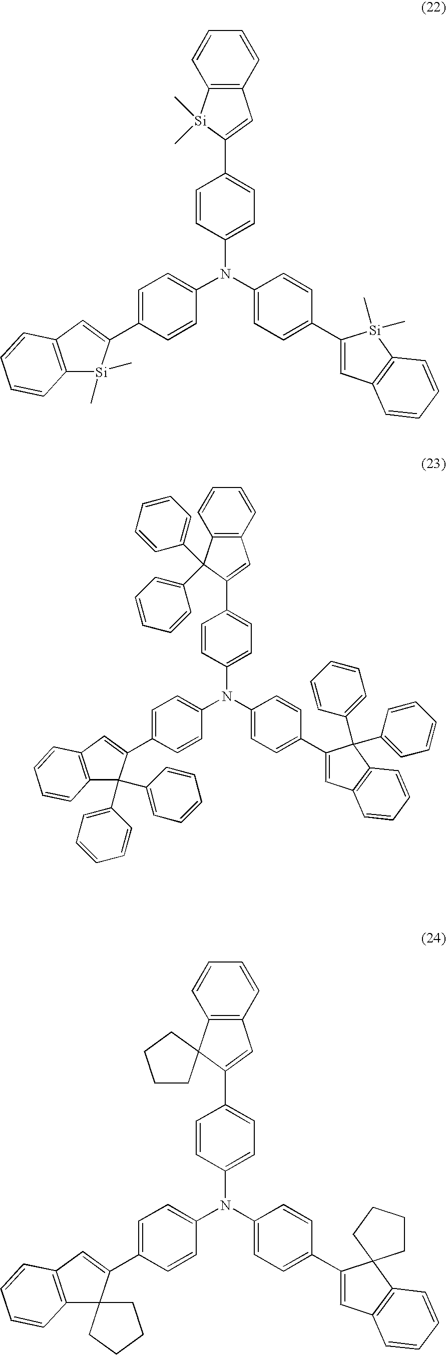

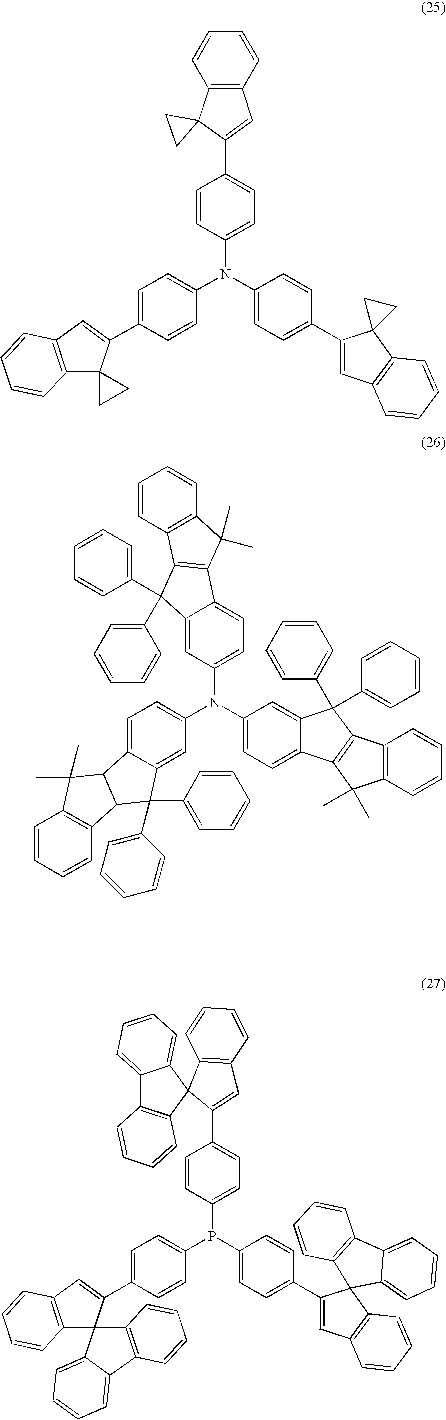

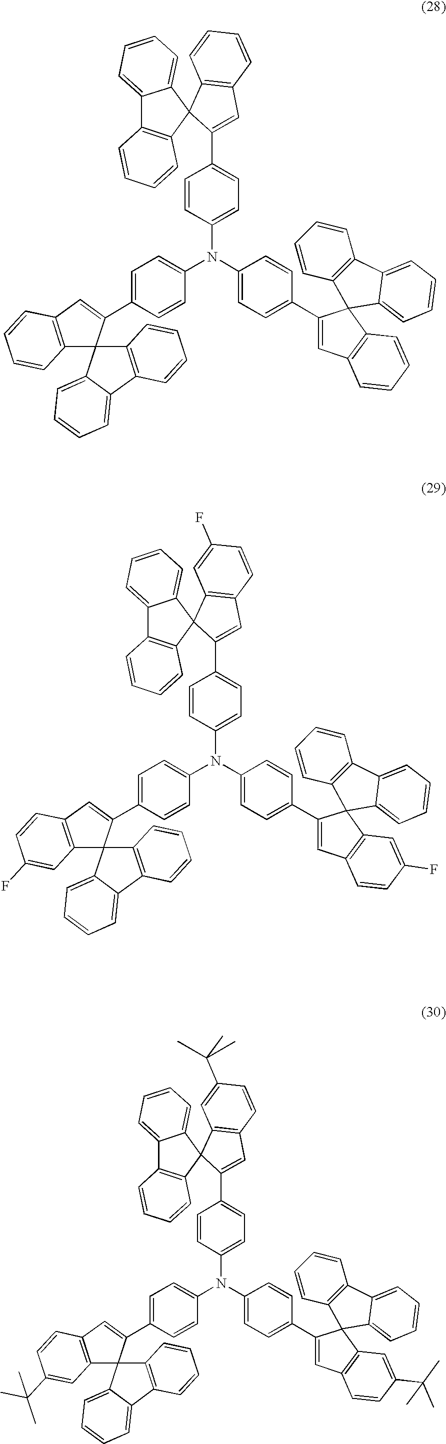

29. The compound according to claim 18, wherein the compound of formula (1) is selected from the structures of formulae (4), (5), (6), (7), (8), (9), (10), (11) and (12) 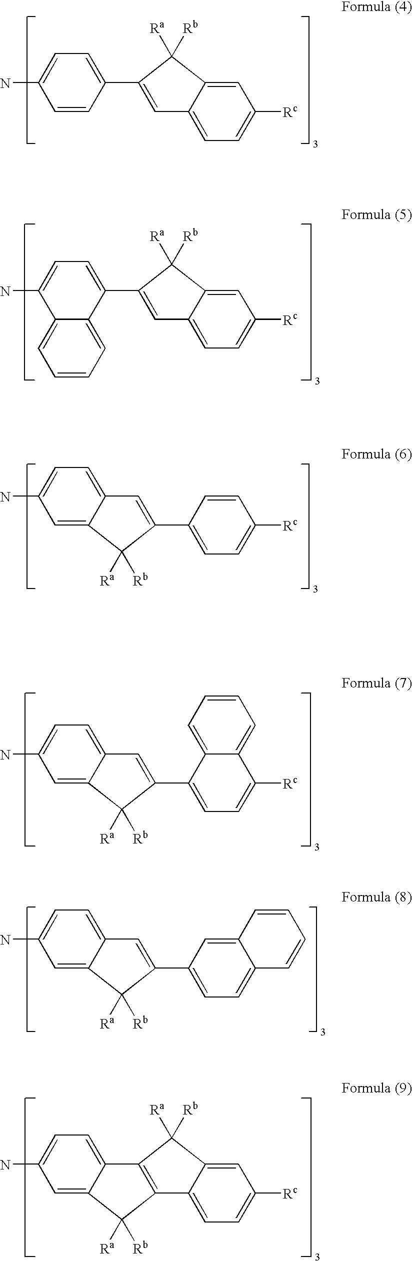 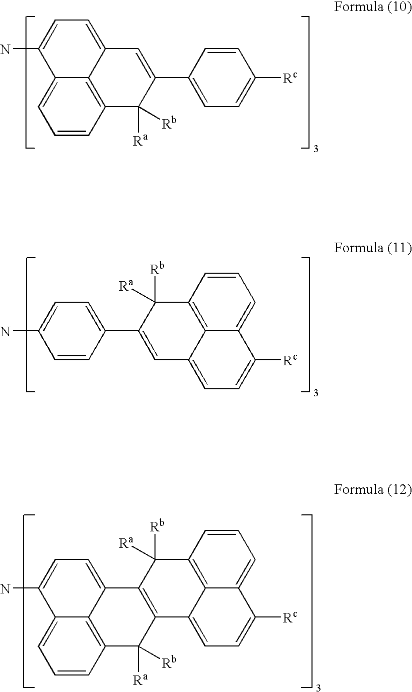
wherein
R^{a} is hydrogen, methyl, F, CF₃ tert-butyl, phenyl, para-tolyl, para-fluorophenyl or para-xylyl,
R^{b} is hydrogen, methyl, F, CF₃, tert-butyl, phenyl, para-tolyl, para-fluorophenyl or para-xylyl, or
R^{a} and R^{b} together form a 2,2′biphenyl and
R^{c} is hydrogen, methyl, F, Br, tert-butyl, phenyl, para-tolyl, or para-xylyl.

30. An organic electronic device which comprises at least one compound of the formula (1) according to claim 18.

31. An organic electroluminescent device comprising cathode, anode and at least one emitting layer, wherein at least one organic layer comprises at least one compound of the formula (1) according to claim 18.

32. The organic electroluminescent device according to claim 31, which further comprises a hole injection layer, hole transport layer, hole blocking layer, electron transport layer and/or electron injection layer.

33. The organic electroluminescent device according to claim 31, wherein the compound of the formula (1) is also used as an emitter and wherein
A is nitrogen at each instance; and
Z is the same or different at each instance and is C(R¹)₂, C(R¹)₂—C(R¹)₂ or C(R¹)₂—NR¹.

34. An organic electroluminescent device according to claim 31, wherein the compound of the formula (1) is used as a hole transport material and wherein
A is nitrogen or phosphorus at each instance; and
Z is the same or different at each instance and is C(R¹)₂, C(R¹)₂—C(R¹)₂, C(R¹)₂—NR¹, O, NR¹ or PR¹.

35. The organic electroluminescent device according to claim 31, wherein the compound of the formula (1) is used as an electron transport material and wherein
A is P═O at each instance; and
Z is the same or different at each instance and is C(R¹)₂, C(R¹)₂—C(R¹)₂, C(R¹)₂—NR¹, C═O, S═O, SO₂, BR¹ or PR¹O.

36. The organic electroluminescent device according to claim 31, wherein the compound of the formula (1) is used as a matrix material or as a hole blocking material in electrophosphorescent device and wherein
A is P═O at each instance; and
Z is the same or different at each instance and is C(R¹)₂, C(R¹)₂—C(R¹)₂, C(R¹)₂—NR¹, C═O, S═O, SO₂, or PR¹O.

37. An organic field-effect transistor (O-FET), organic thin-film transistor (O-TFT), organic integrated circuit (O-IC), organic solar cell (O-SC) and organic laser diode (O-Laser) comprising one or more compounds according to claim 18.
